(19) United States
(12) Patent Application Publication
(10) Pub. No.: US 2012/0195916 A1
(43) Pub. Date: Aug. 2, 2012

(54) METHOD OF TREATING CANCER BY INHIBITING TRIM59 EXPRESSION OR ACTIVITY

(76) Inventor: Jian Wu Xuan, London (CA)

(21) Appl. No.: 13/395,275

(22) PCT Filed: Sep. 10, 2010
(86) PCT No.: PCT/CA2010/001413

§ 371 Date: Apr. 12, 2012

Related U.S. Application Data
(60) Provisional application No. 61/272,315, filed on Sep. 10, 2009.

(51) Int. Cl. A61K 39/395; A61K 38/02; A61K 31/7105; G01N 33/574; C07K 16/18; C40B 30/04; C12Q 1/68; A61K 31/7088; A61P 35/00

(52) U.S. Cl. 424/174.1; 514/44 A; 514/19.3; 514/19.4; 530/389.7; 514/19.5; 506/9; 435/6.12; 435/7.1; 435/7.92

(57) ABSTRACT

A method of treating cancer in a mammal is provided comprising the step of inhibiting TRIM59 expression or activity in the mammal. TRIM59 expression may also be utilized in methods of diagnosing cancer in a mammal.

### FIELD OF THE INVENTION

The present invention generally relates to genes involved in cancer, and more particularly, to the identification of a novel gene of the TRIM gene family, its role in cancer and methods of diagnosis, prognosis and treatment of cancer based on the expression of this TRIM gene.

### BACKGROUND OF THE INVENTION

The TRIM (TRIpartite Motif) family is an evolutionarily conserved gene family comprised of 76 members in the human genome implicated in a number of critical processes including immunity, antivirus, proliferation, transcriptional regulation, neuro-development, cell differentiation and cancer. However, the function of most TRIM family members was surmised only based on computational and sequence analysis mostly derived from their N-terminal RBCC (RING finger, B-box, coiled-coil) domains. The RING (Really Interesting New Gene) finger domain is a cysteine and histidine-rich motif that binds two zinc ions. RING domains are frequently involved in proteolysis acting as E3 ubiquitin ligases and the ubiquitin-proteasome system. Antiviral activity associated with the N-terminal RING-finger E3 ubiquitin ligase has been reported in several members of the TRIM gene family, including the HIV restriction factor TRIM5α variant and the disease-associated proteins TRIM20 (pyrin) and TRIM21, TRIM22, TRIM25, TRIM11, TRIM37, and TRIM39 have also been shown to target retroviruses. B-boxes (1-2) are domains that bind one Zn ion, but their function is unknown. Nine TRIMs were found associated with microtubule binding, suggesting their subcellular compartmentalization, and were characterized as a TRIM subfamily by a unique domain, near the coiled-coil domain and the C-terminus. Recent reports demonstrate TRIM members function in microRNA processing. A large class of TRIM-NHL proteins were characterized with functions as a cofactor for the microRNA-induced silencing complex (miRISC) and thereby enhance the posttranscriptional regulation of several genetically verified microRNA targets. TRIM32 activates microRNAs, targets and ubiquitinylates c-Myc for proteasome-mediated degradation and thereby prevents self-renewal in mouse neural progenitors. An ataxia-telangiectasia group D complementing gene (ATDC) was recently designated as TRIM29, which is elevated in most invasive pancreatic cancers in the Wnt/β-catenin signaling pathway.

Given the foregoing, it would be desirable to elucidate the function of specific TRIM proteins, in order to develop novel diagnostic and treatment methods.

### SUMMARY OF THE INVENTION

Accordingly, methods of diagnosing, prognosing and treating cancer have now been developed utilizing the expression of a novel gene herein referred to as TRIM59.

Thus, in one aspect, a method of diagnosing cancer in a mammal is provided comprising determining the expression or activity of TRIM59 in a biological sample, wherein determination of a level of TRIM59 expression or activity that exceeds a baseline value is indicative of cancer in the mammal.

In another aspect, a method of treating cancer in a mammal is provided, comprising the step of inhibiting TRIM59 expression or activity in the mammal.

These and other aspects of the invention will become apparent in the detailed description and by reference to the following figures.

### BRIEF DESCRIPTION OF THE FIGURES

FIG. 1 illustrates human TRIM59 gene (A) and protein sequences (B), as well as the sequence alignment of human, mouse and rat proteins (C);

FIG. 2 graphically illustrates the “hit-and-run” effect of SV40 Tag oncogene in the tumorigenesis in transgenic (TGMAP) and knock-in (KIMAP) mice including correlative and statistical analyses of Tag IHC signals, for a comparison of WT (wild type mice control) vis PIN (A); WT viz Cancer (WD, MD, PD, B) and in all five gradings (C). n: numbers of mice/numbers of foci studied. * Statistically significant (p<0.01) by student t test. Error bars: ±SD;

FIG. 3 illustrates the procedures used for differential GeneChip (Affymetrix)/cDNA microarray screening for genes associated with SV40 Tag “hit-and-run”effectors;

FIG. 4 illustrates the TRIM gene structure (upper line), TRIM59 cDNA/mRNA structure (second row), the TRIM59 coding region (ORF), 5′ and 3′UTR (untranslated region), two primers (by arrows) for RT-PCR of TRIM59 mRNA, functional domains of RBCC family, and antibodies (TRIM59#71 and #72) shown by arrows. Numbers indicate their location and length. Top line arrows show locations of four shRNA;

FIG. 5 graphically illustrates the results of IHC analyses of TRIM59 protein expression by antibody of TRIM59#72 in transgenic (TGMAP) and knock-in (KIMAP) mouse CaP models;

FIG. 6: graphically illustrates the quantification by densitometry scanning of phosphorylated TRIM59 proteins identified by IMAC column purification and ³²P isotope labeling in NIH3T3 cell cultures;

FIG. 7 graphically illustrates the ELISA quantification and comparison of total TRIM59 protein from a TGMAP tumor and phosphorylation forms (p-Thr and p-Tyr of p-TRIM59) purified by TRIM59 affinity column. Total TRIM59 protein was determined as OD₄₉₂ₙₘ divided by the wet weight (mg) of tissue sample used for affinity column purification, which were also normalized according to volume of sample coated, and the mean of both the first two elution fractions. Extent of phosphorylation of p-Thr and p-Tyr-TRIM59 proteins were determined by calculation of the percentage of their OD₄₉₂ₙₘ values in total TRIM59 proteins (OD₄₉₂ nm), i.e. determined by TRIM59#72 antibody in identical sets of wells in the same plate;

FIG. 8 shRNA knockdown of TRIM59 gene in human prostate cancer cells (DU145) resulted in both S-Phase arrest and cell growth retardation. (A) Graph show results of flowcytometry (FCM) of transient transfection of TRIM59 shRNA (sh1-4) plasmid mixture. (B) Statistical data of (A). (C). Graph show results of flowcytometry (FCM) of stable transfection (selection of Neo^{R} with 200 μg/ml geneticin) of TRIM59 shRNAs. (D). Statistical data of (C). DNA content corresponding to cell division phase was shown in five categories: G0/G1, S, G2/M, 3N (triplets) 4N (quadruplets) and sub-G1(>G1). All graphs show percentages of control. Table shows average and P-value. (E). Real time PCR quantification of RT-PCR products of transient transfection (24 hours and 48 hours) and 2 stable transfectant clones (clones B6, C2, neo^{R}) with TRIM59 shRNAs. cDNA templates (from 1 μg total RNA and 20 μl RT-PCR products) were diluted 1×, 2×, 4× (2 μl, 1 μl, 0.5 μl) separately as gradients to achieve precise C_{T} value determination and comparison. All calculations of real time PCR were conducted according to Invitrogen kit (SYBR GreenET qPCR SuperMix Universal) and the software provided by the ABI 7900HT thermocycler Real Time PCR System. GAPDH were used as internal references. (F). Cell proliferation rate determination of stable transfectant clones (neo^{R}) with TRIM59 shRNA mixtures (sh1-4);

FIG. 9 is a diagram showing differential screening for 24 hours transient transfection Unique gene targets (“unique S24” decrease “S” grey zone). Grey zone means change range within the board of decrease and increase (±10);

FIG. 10 illustrates the structure of PSP-TRIM59 transgene used to establish a transgenic mouse model with PSP94 gene directed up-regulation of mouse TRIM59;

FIG. 11 illustrates cDNA microarray characterization of transgenic mouse PSP94-TRIM59 model showing up-regulation of mouse TRIM59 with interaction in between Ras and pRB signal pathways;

FIG. 12 is a diagram showing the proposed novel signal pathway bridging between Ras oncogene and pRB (SV40Tag binding effector) tumor suppressor gene, mediated by proto-oncogene TRIM59. TRIM59-cyto and TRIM59-nucleus associated bridging genes are listed; and

FIG. 13 graphically illustrates the Gleason graded tissue microarray results obtained on human prostate samples.

### DETAILED DESCRIPTION OF THE INVENTION

Methods of diagnosing, prognosing and treating cancer in a mammal are provided. Methods of diagnosis and prognosis comprise determining the level of TRIM59 expression and/or function as compared with a baseline value. Increased TRIM59 expression or function to the baseline value is indicative of cancer, while a change in the level and/or location of TRIM59 expression/function is indicative of the stage of cancer. A method of treating cancer is also provided in which TRIM59 expression/function is inhibited in the mammal.

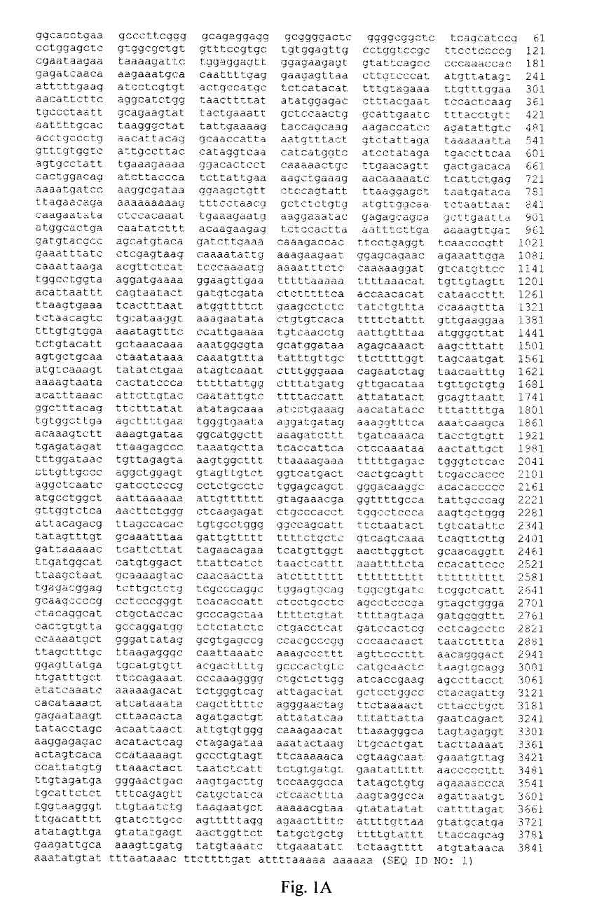

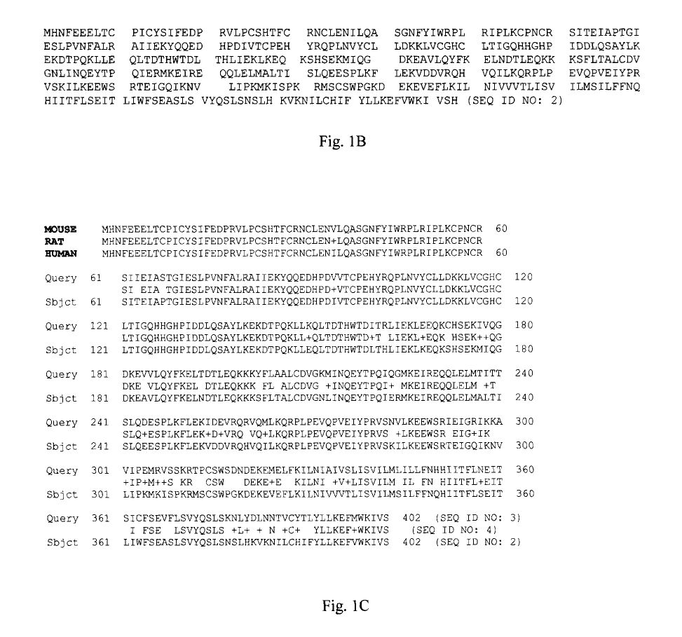

The term “TRIM59” refers to the tripartite motif-containing 59 gene that encodes a trim59 protein. As used herein “TRIM59” encompasses the human gene as well as variants thereof that encode a functional TRIM59 protein including, for example, corresponding genes in non-human mammals. FIG. 1 illustrates the sequence of the human TRIM59 gene (A) and the protein it encodes (B), as well as a sequence alignment between human, rat and mouse TRIM59 (C). The corresponding mouse TRIM59 cDNA and protein sequences are identified by reference to NM 025863, the contents of which are incorporated herein by reference. The corresponding canine protein sequence is identified in XP 545257, also incorporated herein by reference.

The term “cancer” is used herein to refer to a class of diseases in which a group of cells display uncontrolled growth and generally includes carcinoma, sarcoma, melanoma, lymphoma and leukemia, germ cell tumours and blastoma. Examples include, but are not limited to, cancers such as prostate, renal, breast, lung, parotid, brain, thymus, heart, muscle, pancreas, colon, small bowel, stomach, esophagus, bone marrow, spleen, spinal cord, cortex, thyroid, placenta, testis, retina, gastrointestinal, female genital tract including endometrial cancer, cervical cancer and ovarian cancer, bladder, lymph node, adrenal, liver, skin, tongue and mouth (squamous cell cancer), and head and neck mucosal cancer.

The term “mammal” is used herein to refer to both human and non-human mammals.

Increased levels of TRIM59, i.e. to levels greater than that normally found in a healthy individual (a normal or baseline level) is indicative of cancer, e.g. tumorigenesis or tumour initiation. Thus, in the treatment of cancer, it is desirable to down-regulate the expression and/or the function of TRIM59 to inhibit tumorigenesis. As one of skill in the art will appreciate, TRIM59 expression may be inhibited at the nucleic acid level, while TRIM59 function may be inhibited at the protein level.

TRIM59 expression may be inhibited at the nucleic acid level, for example, using anti-sense or RNA-mediated gene silencing technologies. TRIM59-encoding nucleic acid molecules may be used to prepare antisense oligonucleotides against TRIM59-encoding nucleic acid that may be therapeutically useful to inhibit TRIM59 expression. Accordingly, antisense oligonucleotides that are complementary to a nucleic acid sequence encoding TRIM59 according to the invention are also provided. The term “antisense oligonucleotide” as used herein means a nucleotide sequence that is complementary to at least a portion of a target TRIM59 nucleic acid sequence such as that illustrated in FIG. 1.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleiotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and deaza uracils, thymidines, cytosines, adenines, or guanines, 5-tri-fluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linages or short chain heteroatomic or heterocyclic intersugar linkages. For example, the antisense oligonucleotides may contain phosphorothioates, phosphotriesters, methyl phosphonates, and phosphorodithioates. For example, phosphorothioate bonds may link only the four to six 3′-terminal bases, may link all the nucleotides or may link only 1 pair of bases.

The antisense oligonucleotides of the invention may also comprise nucleotide analogs that may be better suited as therapeutic or experimental reagents. An example of such an oligonucleotide analogue is a peptide nucleic acid (PNA) in which the deoxribose (or ribose) phosphate backbone in the DNA (or RNA), is replaced with a polymide backbone which is similar to that found in peptides (P. E. Nielson, et al Science 1991, 254, 1497). PNA analogues have been shown to be resistant to degradation by enzymes and to have extended lives in vivo and in vitro. PNAs also form stronger bonds with a complementary DNA sequence due to the lack of charge repulsion between the PNA strand and the DNA strand. Other oligonucleotide analogues may contain nucleotides containing polymer backbones, cyclic backbones, or acyclic backbones. For example, the nucleotides may have morpholino backbone structures (U.S. Pat. No. 5,034,506). Oligonucleotide analogues may also contain groups such as reporter groups, a group for improving the pharmacokinetic properties of an oligonucleotide, or a group for improving the pharmacodynamic properties of an antisense oligonucleotide. Anti sense oligonucleotides may also incorporate sugar mimetics as will be appreciated by one of skill in the art.

Antisense nucleic acid molecules may be constructed using chemical synthesis and enzymatic ligation reactions using procedures known in the art based on TRIM59 amino acid sequence information such as that provided. The antisense nucleic acid molecules of the invention, or fragments thereof, may be chemically synthesized using naturally occurring nucleotides or variously modified nucleotides designed to increase the biological stability of the molecules or to increase the physical stability of the duplex formed with mRNA or the native gene, e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques well-known in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo.

In another embodiment, RNA-mediated gene silencing technology may be applied to inhibit expression of TRIM59. Application of nucleic acid fragments such as miRNA, siRNA and shRNA that correspond with regions in TRIM59 mRNA may be utilized to selectively block TRIM59 expression. TRIM59 expression is blocked when such RNA fragments bind to TRIM59 mRNA and thereby prevent translation thereof to yield functional TRIM59.

RNA molecules corresponding to a particular region of TRIM59 are made using well-established methods of nucleic acid synthesis including automated systems. Since the structure of the TRIM59 gene is known, fragments of RNA that correspond therewith may readily be made as outlined above with respect to antisense oligonucleotides. The effectiveness of selected RNA molecules to block TRIM59 expression may be confirmed using a TRIM59-expressing cell line. Briefly, a selected RNA molecule is incubated with a TRIM59-expressing cell line under appropriate growth conditions. Following a sufficient reaction time, i.e. for the selected RNA structure to bind with TRIM59-encoding nucleic acid, to result in decreased expression of the TRIM59 DNA, the reaction mixture is tested to determine if such decreased expression has occurred. Suitable RNA structures will prevent processing of the TRIM59 gene to yield functional TRIM59. This may be detected by assaying for TRIM59 function in the reaction mixture.

It will be appreciated by one of skill in the art that RNA fragments useful in the present method may be derived from specific regions of TRIM59-encoding nucleic acid. Moreover, suitable modifications may include, for example, addition, deletion or substitution of one or more of the nucleotide bases therein, provided that the modified RNA fragments retain the ability to bind to the targeted TRIM59 gene. Selected RNA fragments may additionally be modified in order to yield fragments that are more desirable for use. For example, RNA fragments may be modified to attain increased stability in a manner similar to that described for antisense oligonucleotides.

TRIM59 function or activity may be inhibited in any one of a number of ways to treat cancer in a mammal. At the outset, synthetic inhibitors of TRIM59, such as chemical inhibitors, may be determined, for example, using assays designed to detect reduced TRIM59 activity or assays designed to determine binding affinity of a candidate compound to TRIM59. TRIM59 function may also be inhibited using naturally- or non-naturally occurring compounds such as proteins, including but not limited to, immunological inhibition using antibodies designed for this purpose. Such immunological techniques are described in more detail herein.

Thus, administration to the mammal of an inhibitor effective to at least reduce TRIM59 expression or function is effective to treat cancer in a mammal. As set out, effective inhibitors may include oligonucleotides, proteins, antibodies and chemical inhibitors. As one of skill in the art will appreciate, the administrable route of the inhibitor will vary with the condition being treated, and the target tissue. Dosages of inhibitors effective to reduce TRIM59 expression or function may readily be determined using assays established in the art.

The inhibitor may be administered alone or as a composition in conjunction with a pharmaceutically acceptable adjuvant. The expression “pharmaceutically acceptable” means acceptable for use in the pharmaceutical and veterinary arts, i.e. not being unacceptably toxic or otherwise unsuitable. Examples of pharmaceutically acceptable adjuvants include diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Creams, lotions and ointments may be prepared for topical application using an appropriate base such as a triglyceride base. Such creams, lotions and ointments may also contain a surface active agent. Aerosol formulations, for example, for nasal delivery, may also be prepared in which suitable propellant adjuvants are used. Other adjuvants may also be added to the composition regardless of how it is to be administered, for example, anti-microbial agents may be added to the composition to prevent microbial growth over prolonged storage periods.

In another aspect, a method of diagnosing cancer in a mammal is provided comprising determining the level of TRIM59 expression or function in a biological sample from the mammal. Determination of a level of TRIM59 expression or function that exceeds a baseline mean value in normal healthy mammals is indicative of cancer, e.g. tumorigenesis, in the mammal.

In the diagnostic aspects of the invention, a biological sample is obtained from a mammal that is suitable to quantify either the expression level of TRIM59 (TRIM59 or a naturally occurring variant thereof), e.g. the level of TRIM59 protein or the level of TRIM59-encoding nucleic in the sample. Suitable biological samples for this purpose include tissue, blood, saliva, urine, semen, hair, skin and cerebrospinal fluid. The sample is obtained from the mammal using methods conventional for the sample type. Many of these samples can readily be obtained in a non-invasive manner. Cerebrospinal fluid is obtained using the spinal tap procedure. The amount of biological sample required must be sufficient to allow quantification of TRIM59 protein or TRIM59-encoding nucleic acid therein. For example, an amount of about 5 ug protein is generally needed for TRIM59 quantification, while about 10 ng nucleic acid is generally needed for TRIM59 nucleic acid quantification.

In order to quantify TRIM59 protein content in a biological sample, the protein fraction is first isolated therefrom using standard isolation and fractionation techniques including lysis/centrifugation, precipitation and separation using, for example, electrophoresis and chromatography such as HPLC and affinity. Quantification of TRIM59 may then be conducted in a number of ways as will be appreciated by one of skill in the art. TRIM59 may be isolated using a separation method and then quantified against standards. Immunological techniques, for example, may also be employed to identify and quantify TRIM59 either on its own or in conjunction with a separation technique. A TRIM59 primary antibody may be used in an affinity column to separate TRIM59 from a sample and a detectably labeled secondary antibody may be used for identification purposes. Also, detectably labeled (e.g. fluorescent, colorimetric, radioactive) TRIM59 antibody, or a related compound, may be linked to TRIM59 exposed in the sample or separated from a sample and quantified. Methods of making antibodies for use in the diagnostic methods are detailed below.

In another embodiment, TRIM59 in a biological sample may be quantified by measuring the amount of TRIM59-encoding nucleic acid within the sample. For example, mRNA copy number may be measured by techniques well-established in the art. Briefly, mRNA copy number may be determined using PCR, for example, one-step real-time PCR in which TRIM59 forward and reverse primers are used to amplify TRIM59 mRNA for quantity determination against pure TRIM59 mRNA standards.

Having determined the level of TRIM59 expression or activity in a biological sample obtained from a mammal, a comparison with a control (baseline) value determined to exist in a normal, undiseased state, is made. It has been determined that an increase in the expression of TRIM59, depicted by an increase in TRIM59 nucleic acid, protein or protein function, from a normal or baseline value is indicative of cancer.

In prognostic aspects of the invention, the level and/location of TRIM59 expression or activity may also be indicative of the stage of cancer. For example, up-regulation of TRIM59 from the baseline value is indicative of tumorigenesis. However, at later advanced stages of cancer, TRIM59 expression is decreased from the level that occurs during tumorigenesis. In one embodiment, TRIM59 expression may be decreased to the baseline level or less in an advanced stage of cancer. Accordingly, having diagnosed tumour initiation in a mammal, a subsequent decrease in TRIM59 expression would evidence progression of the disease to an advanced state.

The determination of phosphorylated forms of TRIM59 protein in a biological sample may also be indicative of cancer, and the nature of the phosphorylation may be used to determine the stage of cancer, e.g. tumorigenesis versus an advanced stage of cancer. For example, an increase in the level of phosphorylated serine/threonine (pS/pT)TRIM59 protein may be indicative of tumorogenesis, while an increase in the level of phosphorylated tyrosine (pY) TRIM59 may be indicative of an advanced stage of cancer. Phosphorylated forms of TRIM59 may be detected immunologically as described in the specific examples herein.

Antibodies to TRIM59 proteins, including phosphorylated forms thereof, are provided in another aspect of the invention. Such antibodies are useful in diagnostic, prognostic and treatment methods of the invention as described above. Conventional methods may be used to prepare the antibodies including polyclonal antisera or monoclonal antibodies. To produce polyclonal antibodies, a mammal, (e.g. a mouse, hamster, or rabbit) can be immunized with an immunogenic form of the protein which elicits an antibody response in the mammal, e.g. a full-length TRIM59 sequence such as the sequences set out in FIG. 1C, a C-terminal fragment of TRIM59, or an N-terminal fragment of the TRIM59. Techniques for conferring immunogenicity on a peptide are well known in the art and include, for example, conjugation to carriers. The peptide can be administered in the presence of adjuvant. The progress of immunization can be monitored by detection of antibody titers in plasma or serum. Standard ELISA or other immunoassay procedures can be used with the immunogen as antigen to assess antibody levels. Following immunization, antisera can be obtained and, if desired, polyclonal antibodies isolated from the sera.

To produce monoclonal antibodies, antibody-producing cells (lymphocytes) are harvested from an immunized animal and fused with myeloma cells by standard somatic cell fusion procedures to form immortal hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al., Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with a selected TRIM59 peptide and the monoclonal antibodies can be isolated.

The term “antibody” as used herein is intended to include fragments thereof which also specifically react with a TRIM59 protein according to the invention. Antibodies may be fragmented using conventional techniques and the fragments screened for utility in the same manner as described above. For example, fragments can be generated by treating an antibody with pepsin. The resulting fragments can be further treated to reduce disulfide bridges.

Chimeric antibody derivatives, i.e., antibody molecules resulting from the combination of a variable non-human animal peptide region and a constant human peptide region are also contemplated within the scope of the invention. Chimeric antibody molecules can include, for example, the antigen binding domain from an antibody of a mouse, rat, or other species with a constant human peptide region. Conventional methods may be used to make chimeric antibodies containing the immunoglobulin variable region which recognizes a TRIM59 protein of the invention (See, for example, Morrison et al., Proc. Natl. Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Monoclonal or chimeric antibodies specifically reactive with a TRIM59 protein of the invention as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al, Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great. Britain).

Embodiments of the invention are described in the following specific example which is not to be construed as limiting.

### Example 1

### Materials and Methods PSP94 Gene Directed TRIM59, TGMAP and KIMAP GEM-CaP Models, Histology and Pathology:

The PSP94-TRIM59 mouse model was established using a transgene comprised of the 3.842 bp PSP94 promoter/enhancer region including the first exon (53 nucleotides), the complete mouse TRIM59 ORF with the stop codon replaced by FLAG tag (MDYKDDDDK), SV40 splicing sequences and SV40 poly A sequences (from pBALCAT, Clontech). All sequence modifications were introduced by PCR cloning and confirmed by DNA double stranded sequencing. Transgenic mice were prepared in London's (Ontario) transgenic targeting facility and three breeding lines were confirmed using appropriate primers by a quick tail PCRF procedure. The TGMAP and KIMAP models were similarly prepared as described in Gabril et al. (Mol. Ther., 11: 348-362, 2005).

Protocols and standards for mouse micro-dissection, anatomical, pathological and histological grading were performed using established techniques as previously reported (for example, in Gabril et al., 2005; see full citation above) For each mouse at different age groups (weeks), ventral (VP) and dorsolateral (DLP) prostate lobes were processed for formalin fixed and H&E staining slides separately. Histo-pathological classifications were performed according to the standard of the following five histological grading categories: Hyperplasia (Hyp), mouse PIN (prostatic intraepithelial neoplasia) (mPIN), well differentiated adenocarcinoma (WDCaP), moderately differentiated adenocarcinoma (MDCaP) and poorly differentiated carcinoma (PDCaP). All grading determination and analyses were performed blindly by at least two authors independently. All animal experiments were conducted according to standard protocols approved by the University of Western Ontario Council on Animal Care (UCAC).

cDNA Microarray (GeneChip, Affymetrix) Analysis:

Total cellular RNA from micro-dissected prostate tissues was extracted and purified by using the TRIzol (Invitrogen, Burlington, ON) and RNeasy Mini Kit (Qiagen, Valencia, Calif.). All purified total RNA preparations from individual mice were assessed with an Agilent 2100 Bioanalyzer (Agilent, Palo Alto, Calif.) separately before pooling. cDNA and cRNA syntheses were performed as per GeneChip Expression Analysis Technical Manual protocols (Affymetrix, Santa Clara, Calif.). All chip experiments were performed at the London Regional Genomics Centre, London, Ontario, Canada. The quality of the labeled target was assessed on a Test 3 array prior to hybridization. Prostate samples from transgenic models were compared with the wildtype in Affymetrix GeneChips of MG_U74Av2 MOE430A or MOE 430 2.0. For human cell line GeneChip analysis, HGU133 Plus 2 chip array was utilized. Gene expression levels of samples were normalized and analyzed using standard software (Microarray Suite, Data Mining Tools, GeneSpring) provided by Affymetrix available at http://www.affymetrix.com/analysis/go. Classification was determined by NCBI/Unigene and PubMed publications.

### Semi-Quantitative Rt-PCR, Real Time PCR and Northern Blotting:

Semi-quantitative RT-PCR analysis was performed based on the size and relative quantity according to reported procedures that PCR amplification samples were taken from PCR cycles. Wildtype (Wt) mouse prostate, NIH 3T3 and GAPDH (glyceraldehyde-3-phosphate dehydrogenase) were used as controls. Real time PCR was conducted according to the Invitrogen kit (SYBR GreenET qPCR SuperMix Universal). All tests were performed with 3 dilution gradients of the templates cDNA. Results were calculated according to software provided by the ABI 7900 Real Time PCR System. Oligonucleotide DNA primer pairs are listed in Table 1.

TABLE 1
Supplemental Table s-1 List of PCR primers used in this study.

| | | |
|---|---|---|
| GAPDH F226 | 5-′GAAGGTGAAGGTCGGAGTC-3′ | Real time RT-PCR |
| | (SEQ ID NO: 5) | |
| GAPDH R226 | 5-′GAAGATGGTGATGGGATTTC-3′ | RT-PCR |
| | (SEQ ID NO: 6) | |
| TRIM59-3F | 5-′TCACCTGCCCTGAACATTAC-3′ | RT-PCR |
| | (SEQ ID NO: 7) | |
| TRIM59-3R | 5′-CAGCTTCCTTATCGCCTTG-3′ | RT-PCR |
| | (SEQ ID NO: 8) | |
| GAP116-F | 5′-GGTCTCCTCTGACTTCAACA-3′ | RT-PCR |
| | (SEQ ID NO: 9) | |
| TRIM59sh1top | 5′-GATCC GAAGAGTCTCCACTTAAAT TTCAAGAGA ATTTAAGTGGAGACTCTTCTT A-3′ | |
| | (SEQ ID NO: 10) | |
| TRIM59sh1b | 5′--AGCTT AAGAAGAGTCTCCACTTAAAT TCTCTTGAA ATTTAAGTGGAGACTCTTC G-3′ | |
| | (SEQ ID NO: 11) | |
| TRIM59sh2top | 5′--GATCC TATGGTTTTCTGAAGCCTC TTCAAGAGA GAGGCTTCAGAAAACCATATT A-3′ | |
| | (SEQ ID NO: 12) | |
| TRIM59sh2b | 5′--AGCTT AATATGGTTTTCTGAAGCCTC TCTCTTGAA GAGGCTTCAGAAAACCATA G-3′ | |
| | (SEQ ID NO: 13) | |
| TRIM59sh3top | 5′--GATCC TGTCAACCTGAATTGTTTA TTCAAGAGA TAAACAATTCAGGTTGACATT A-3′ | |
| | (SEQ ID NO: 14) | |
| TRIM59sh3b | 5′--AGCTT AATGTCAACCTGAATTGTTTA TCTCTTGAA TAAACAATTCAGGTTGACA G-3′ | |
| | (SEQ ID NO: 15) | |
| TRIM59sh4top | 5′--GATCC ATGGGCTTATTCTGTACAT TTCAAGAGA ATGTACAGAATAAGCCCATTT A-3′ | |
| | (SEQ ID NO: 16) | |
| TRIM59sh4b | 5′--AGCTT AAATGGGCTTATTCTGTACAT TCTCTTGAA ATGTACAGAATAAGCCCAT G-3′ | |
| | (SEQ ID NO: 17) | |
| u7F2 | 5′--GTT CAC AGC CAT TGA AAT CCC C-3′ | RT-PCR |
| | (SEQ ID NO: 18) | |
| U7r2 | 5′-CAA ACT CAG CCT CCT GGC AAA G-3′ | RT-PCR |
| | (SEQ ID NO: 19) | |
| U7GST-f | 5′-GGGTT GGATCC ATGCACAATTTTGAGGAGGAGTTA ACG-3′ | GST-TRIM59 |
| | (SEQ ID NO: 20) | |
| u7GST-r | 5′-GGGAA GGATCC AAGGCGAGTGATATC TGTCC-3′ | GST-TRIM59 |
| | (SEQ ID NO: 21) | |
| U7GST2-n | 5′-GGGTT GGATCC CCT CGA GTA AGC AAT GTA-3′ | GST-TRIM59 |
| | (SEQ ID NO: 22) | |
| u7GST2-c | 5′-GGGAA GGATCC TCA ACG AGA AAC TAT TTT C-3′ | GST-TRIM59 |
| | (SEQ ID NO: 23) | |
| U7orf.n.F | 5′ GGGTT AAGGAGT CCTGCTTTGT CACC ATG GCA CCC AAG AAG AAG AGG | TRIM59 transgene |
| | AAGGTG CACAATTTTG AGGAGGAGTT AACG 3′ | |
| | (SEQ ID NO: 24) | |
| prFLAG | 5′-CTT ATC GTC GTC ATC CTT GTA ATC-3′ | TRIM59 transgene |
| | (SEQ ID NO: 25) | |
| U7flagr | 5′-CACAATTTTG AGGAGGAGTT AACG 3′ | RT-PCR for TRIM59 |
| | (SEQ ID NO: 26) | transgene |
| U7.959.F | 5′-ATT TAT CCT CGA GTA AGC AAT GTA-3′ | RT-PCR for TRIM59 |
| | (SEQ ID NO: 27) | transgene |
| U7TGSV-f | 5′-GGGTT CTCGAG ATCTTTGTGA AGGAACCTTAC-3′ | TRIM59 transgene |
| | (SEQ ID NO: 28) | |
| U7tgsvpe-r | 5′-GGGAA GGTACC TCTAGA ATCGATCCAGAC ATGATAAG-3′ | TRIM59 transgene |
| | (SEQ ID NO: 29) | |
| U7toe1 | 5′-TGG TCT TCT TGC TGG TAC-3′ | Genotyping of tg |
| | (SEQ ID NO: 30) | TRIM50 |
| MPR36 | 5′-GGC AAC AGC GTG TCA AAG--3′ | TG, KMAP, tg |
| | (SEQ ID NO: 31) | TRIM59 genotyping |
| PrSVtag | 5′-CAA GAC CTA GAA GGT CCA TTA GC-3′ | TG, KIMAP |
| | (SEQ ID NO: 32) | genotyping |
| Rac2.F | 5-CCATCGCTTTGGGGAGT-3 | Real time RT-PCR |
| | (SEQ ID NO: 33) | |
| Rac2.R | 5-ACAGGCCGGGGTTTGC-3 | Real time RT-PCR |
| | (SEQ ID NO: 34) | |
| Fos.F | 5-CCGAAGGGAACGGAATAA-3 | Real time RT-PCR |
| | (SEQ ID NO: 35) | |
| Fos.F | 5-CTGGGAAGCCAAGGTCAT-3 | Real time RT-PCR |
| | (SEQ ID NO: 36) | |
| Gpr120.F | 5-CCTTCACGTTTGCCAACTC-3 | Real time RT-PCR |
| | (SEQ ID NO: 37) | |
| Gpr120.R | 5-GCACTGGTGGGCTTTCC-3 | Real time RT-PCR |
| | (SEQ ID NO: 38) | |
| Gpr18.F | 5-TGAAGCCCAAGGTCAAGG-3 | Real time RT-PCR |
| | (SEQ ID NO: 39) | |
| Gpr18.R | 5-CAGGACGGCAAAGCAGAT-3 | Real time RT-PCR |
| | (SEQ ID NO: 40) | |
| Pla2g2a.F | 5-ATGAAGGTCCTCCTGCTGC-3 | Real time RT-PCR |
| | (SEQ ID NO: 41) | |
| Pla2g2a.R | 5-GGGGAATCCTTTGCCACC-3 | Real time RT-PCR |
| | (SEQ ID NO: 42) | |
| Sgpp2.F | 5-AGTGTAAGCAACGCACGACG-3 | Real time RT-PCR |
| | (SEQ ID NO: 43) | |
| Sgpp2.R | 5-GCCAAGCAATGACGAAAGG-3 | Real time RT-PCR |
| | (SEQ ID NO: 44) | |
| Styk1.F | 5-GTGCCTGAACTGTATGC-3 | Real time RT-PCR |
| | (SEQ ID NO: 45) | |
| Styk1.R | 5-AGCCCTTGGGACTGG-3 | Real time RT-PCR |
| | (SEQ ID NO: 46) | |
| Ccnb1-rs1.F | 5-TAAAGCCCTACCAAAACC-3 | Real time RT-PCR |
| | (SEQ ID NO: 47) | |
| Ccnb1-rs1.R | 5-CCCCATCATCTGCGTC-3 | Real time RT-PCR |
| | (SEQ ID NO: 48) | |
| P107.F | 5-AATGGTCCAGGAAACACG-3 | Real time RT-PCR |
| | (SEQ ID NO: 49) | |
| P107.R | 5-TGGCTGCAAATCGAGAA-3 | Real time RT-PCR |
| | (SEQ ID NO: 50) | |
| Rbbp8.F | 5-AGACACCGATTTCGCTAC-3 | Real time RT-PCR |
| | (SEQ ID NO: 51) | |
| Rbbp8.R | 5-TTTTGGGACGAGGACTAA-3 | Real time RT-PCR |
| | (SEQ ID NO: 52) | |
| Rbbp4.F | 5-CAGCAGTAGTGGAGGACG-3 | Real time RT-PCR |
| | (SEQ ID NO: 53) | |
| Rbbp4.R | 5-TATGGGATTCAAAGGAGTG-3 | Real time RT-PCR |
| | (SEQ ID NO: 54) | |
| Trp53bp1.F | 5-AAGTTGGGGAATAGGTTGA-3 | Real time RT-PCR |
| | (SEQ ID NO: 55) | |
| Trp53bp1.R | 5-AGGCTTTGCAGAATGGA-3 | Real time RT-PCR |
| | (SEQ ID NO: 56) | |
| CHG-A.F | 5′-AGA GGA CCA GGA GCT AGA GAG-3′ | Real time RT-PCR |
| | (SEQ ID NO: 57) | |
| CHG-A.R | 5′-TAA TAG TCA GGA GTT CTC GGC-3′ | Real time RT-PCR |
| | (SEQ ID NO: 58) | |

Expression of Recombinant GST-Mouse TRIM59 Fusion Protein in E. coli, Generating of Mouse TRIM59 Antibodies:

A full length cDNA clone of mouse TRIM59 (NM_{—}025863, 2858 bp) was purchased from Invitrogen (MGC IRAV 4017983). GST-TRIM59#U71 and #U72 constructs, containing an N-terminal fragment (163 aa from cDNA sequence 127-616) and a C-terminal fragment (126aa, from cDNA sequence 961-1338) separately, were cloned by PCR (primer pairs see Table 1) into pGEX-2T expression vectors (GE-Amersham, Montreal, Que). All clones of PCR fragments of TRIM59 in pGEX2T expression vector were confirmed by double stranded DNA sequencing. GST-TRIM59 proteins were characterized by SDS-PAGE with GST protein as control. Purification of GST-fusion proteins in E. coli lysates by a GST affinity column (Glutathione Sepharose 4B from GE-Amersham) was performed following the manufacturer's recommended procedures. Approximately 1.5 mg of purified GST-TRIM59 proteins were immuned to each rabbit according to University (UCAC) standard protocols. Rabbit antiserum was first tested to recombinant GST-proteins, and then purified by protein A Sepharose resin (GE-Amersham) according to the manufacturer's instruction.

### Immunohistochemistry (IHC).

Standard ABC (Avodin Biotin Complex) protocol was used as previously reported (Wirtzfeld et al. Cancer Res., 65: 6337-6345, 2005). Briefly, deparaffinized, and rehydrated sections were treated with 0.3% hydrogen peroxide in methanol for 15 minutes at room temperature to block endogenous peroxidase activity. Antigen retrieval was done by autoclaving in 10 mM citrate buffer pH 6.0 for 5 minutes. After blocking with 10% goat serum in phosphate buffered saline (PBS), sections were incubated with first antibody and reaction was at 4° C. overnight. All first antibodies used for this study were tested for optimal dilutions and a moderate dilution was determined for the best differentiation of tumor samples. All IHC slides were counterstained by hematoxylin.

### Cell Culture and ³²P Labeling in Cultured Cells:

Mouse fibroblast cell line NIH 3T3, HEK293, human prostate cancer cell lines DU145, PC3 and LNCaP cell lines (rat prostate endothelial cells of 8-wk-old Copenhagen male rats). were maintained in RPMI 1640 medium or DMEM (Invitrogen/Gibco) supplemented with 10% fetal bovine serum (FBS). All cell cultures were maintained at standard cell culture conditions (37° C., 5% CO₂ in a humidified incubator). Cultured cells were lysed at 2−3×10⁷ cell/ml lysate buffer. For ³²P labeling of total cellular phosphorylated proteins in cultures cells, standard protocols were followed. In brief, phosphorus −32 (H₃PO₄, HCl free, 400-800mCi/ml, MP Biochemicals, Irvine, Calif.) were added to 80% confluent NIH 3T3 cells at a concentration of 0.5 mCi/ml (5mCi/10 ml) of DMEM media with out phosphate (Invitrogen/Gibco) supplemented with 10% FBS (without dialysis against water) and labeled for 3-4 hours. Labeled cultured cells were lysed and immunoprecipitated with TRIM59 antibodies according to IP procedures.

### Detection of Phosphoprotein by IMAC (Immobilized Metal Affinity Chromatography) Column in Cell Culture and Mouse Prostate Tissues:

Approximately 2−3×10⁷ NIH3T3 cells were lysed for each column (from PhosphoProtein kit, Qiagen, Montreal Que and Phosphoprotein Enrichment kit, Clontech, Calif.), and the total cellular phosphoproteins were purified. All solutions and experimental procedures were performed according to the manufacturer's instructions. For detection and semi-quantitative determination of phosphorylated TRIM59 proteins in GEM-CaP mice by IMAC separation procedures, tumor samples were dissected and snap-frozen in liquid nitrogen. Tumor sample lysates were prepared in the lysis buffer and proteinase inhibitors from the Qiagen kit, homogenized and cleared by repeated high speed centrifugation. All cleared tissue lysates were first titrated by BioRad protein assay and then diluted to 25 ml with lysis/binding buffer provided by the kit. Samples were taken from every separation procedure (named “before”, “pass”, “wash”, eluate “E1”, “E2” . . . ) and were resolved in 10% SDS-PAGE and transferred to PVDF membranes. All purified proteins were concentrated or desalted by a centrifugal ultra-filtration tube (Ultrfree-0.5, 5KUMWL, Millipore).

Prostate tissue lysates (0.3-0.6 mg wet weight/ml) were prepared by homogenizing in a lysis buffer (50 mM Tris/HCl, pH 7.8, 150 mM NaCl, 1.0% Triton X-100, 1 mM EDTA, 1 mM EGTA, 1 mM PMSF, 2 mM Na₃VO₄, 10 mM β-glycerophosphate, 5 mM Sodium pyrophosphate and proteinase inhibitor cocktail (×100). Tumor tissues or cultured cells were lysed by homogenization and cleared by repeated high speed centrifugation at 4° C. for 20 minutes. All cleared tissue lysates were first titrated by BioRad protein assay then diluted to 25 ml with lysis/binding buffer provided by the kit.

### Immunoprecipitation (IP) by Immobilized Antibody:

About 200 μg of protein A column purified IgG were coupled and immobilized onto 200 μl of gel slurry (AminoLink Plus Coupling Gel) following manufacturer's instruction (Seize® Primary Mammalian IP kit). Samples were taken each time from washing of the column when started, from the last washing and elution fractions for Western blotting analysis, only very weak antibodies of the coupling rabbit antiserum were observed.

### Establishment of the Mouse TRIM59 Immuno-Affinity Column:

Approximately 2 ml of rabbit antiserum of mouse recombinant TRIM59 were purified by Protein A column (GE-Amersham), and were used as a ligand to covalently conjugate to pre-packed NHS (N-hydroxy-succinimide)-activated Sepharose column (1 ml, HiTrap NHS-activated HP, from GE-Amersham). The manufacturer's instructions were followed for all subsequent steps of the preparation of HiTrap NHS-activated HP, activating, coupling, blocking and washing.

### Transient and Stable Transfection of Cultured Cells

This was performed using Lipofectamin 2000 (Invitrogen/Gibco) according to the manufacturer's protocol. Approximately 1×10⁶ cells were inoculated in 60 mm Petri dish, grown overnight, and transfected with approximately 2 μg of plasmid DNA.

### ECL Western Blotting:

Standard ECL Western blotting experiments (Amersham, Calif.) were performed by SDS-PAGE, using 10% polyacrylamide gels, and then transferred to PVDF membranes. Column eluates containing protein peaks were concentrated, electrophoresed on 10% SDS-PAGE gels, and transferred to ECL membranes for Western blot analyses. Dilution of antibodies used for this study was a rabbit antiserum to mouse GST-TRIM59 #71 and #72 at a dilution of (1:1000) and goat anti-rabbit or mouse HRP conjugate (1:1000, CalBiochem). Antibodies against phosphoproteins of p-threonine (P-Thr-Polyclonal, #39381, used at 1:1000 dilution) and p-tyrosine (monoclonal AB #9411, used at 1:2000 dilution) were all from Cell Signaling, NEB, MA.

### ELISA Quantification of Mouse TRIM59 Proteins and Extent of Phosphorylation Forms in Tumor Tissues and Cultures:

Standard direct ELISA protocols were followed. In brief, duplicate or triplicate samples from affinity purified TRIM59 proteins were coated overnight at 4° C. with different concentrations in a buffer of 14 mM Na₂CO₃, 70 mM NaHCO₃, pH 9.6 in a 96 well plate (NUNC ImmunoPlate II, with MxiSorb surface from VWR Canada). Plates were blocked in 1.5% BSA, PBS, 0.05% Tween 20, reacted with first antibody and second HRP-conjugated antibody, each for 1 hour. Color reaction was by 4 mg/ml OPD (o-phenylenediamine dihydrochloride, Sigma) and 0.5% H₂O₂ and the OD (optical density) was measured at 492 nm in a 94 well reader (Multiskan EX, Thermo, Finland).

### Flow Cytometry (FCM)

This was performed on the harvested transfectant cells and stained with propidium iodide according to the protocol of Beckman Coulter—Coulter DNBA Prep reagents kit. DNA histograms were measured and analyzed using the EPICS XL-MCL flow cytometer (Beckman Coulter Electronics, Hialeah, Fla.).

### Cell Proliferation Rate

This was determined by counting cells in a times course test in 60 mm culture Petri dishes (each inoculated with 1×10⁵ cells).

In Vivo Cell Proliferation Test: Brdu (5-bromo2′-deoxy-uridine, from Sigma) Labeling and Immunohistochemistry

These were performed according to the protocol provided by Chemicon. Each mouse was injected with 0.1 mL of BrdU (10 mg/ml in PBS) per gram of body weight (30-100 mg/kg). Prostate samples were collected and fixed in formalin. IHC was performed using a monoclonal antibody of BrdU (Sigma 1: 300) following DNA denaturation and trypsin treatments.

### Gleason Graded Tissue Microarray Analysis

Slides (H&E) from radical prostatectomy specimens (from 2006 to 2008) were obtained from the Vancouver General Hospital. The patients had no prior treatment. Benign and cancer sites were identified and marked in donor paraffin blocks using matching H&E reference slides. Tissue microarray (TMA) was constructed using a manual tissue micro arrayer (Beecher Instruments, Silver Spring, Md.). Each block marked for benign and cancer was sampled twice with a core diameter of 1 mm arrayed in rectangular pattern with 0.7 mm between the centers of each core, creating a duplicate TMA layout and ordered by histopathology of specimen and tumor's Gleason grade (as set out in Table 2 in the Results section). The number of patients in this TMA is 88 with a total of 176 cores. The TMA paraffin block was sectioned into 0.5 micrometer sections and mounted on the positively charged slides. Immunohistochemical staining was conducted by Ventana autostainer model Discover XT™ (Ventana Medical System, Tuscano, Ariz.) with enzyme labelled biotin streptavidin system and solvent resistant DAB Map kit by using 1/25 concentration of TRIM 59 Rabbit polyclonal antibody (provided by Dr. Jim Xuan). TMA was scanned by Bliss Digital imaging system using 20× objective, from Bacus Laboratories INC, Centre Valley Pa., and stored in the Prostate Centre Saver; http//bliss.prostatecentre.com. A value on a four-point scale was assigned to each core. Descriptively, 0 represents no staining by any tumor cells, 1 represents a weak stain, 2 represents a stain of moderate intensity in a convincing number of cells, and 3 represents strong immunoreactivity by a sufficient number of cells.

### Statistical Analysis:

Student's t tests and one-way ANOVA were used to analyze the data with p<0.05 considered to be statistically significant. All graphs with error bars were generated by Microsoft Excel or SigmaPlot 2000 programs.

### Results

Determination of the Globe Gene Profile of Tumorigenesis Correlating with the SV40 Tag “Hit-And-Run” Effects In Vivo in GEM-Cap Models by Differential cDNA Microarray/GeneChip Screening:

To determine the initiation effects of SV40 Tag oncogenesis, a systematic immunohistochemistry (IHC) study was performed in SV40 Tag oncogene directed transgenic and knock-in mouse KIMAP and TGMAP tumors. Tumors were assigned to 5 main grades, i.e. normal (WT) with hyperplasia, PIN, WD, MD and PD CaP. A total of 113 foci from 34 mice with ages ranging from 6 to 91 weeks were examined. Five wildtype (WT) mice were used as controls.

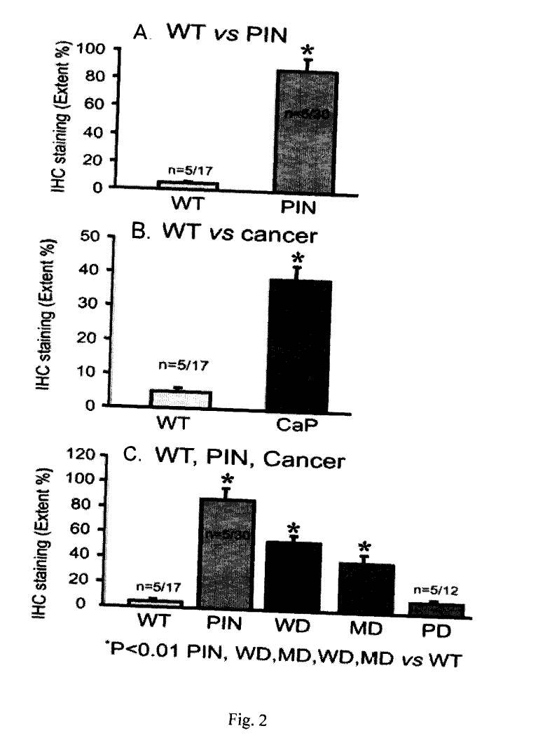

A high SV40 Tag expression in foci of hyperplasia, PIN and WD CaP, were observed, and lower levels of expression in foci of MD CaP and PD CaP reminiscent of a “hit-and-run” effect of SV40 Tag in the GEM-CaP model (FIG. 2). All SV40 Tag IHC signals in prostate tissues were detected in the nucleus, while no staining was observed in wildtype mice or prostate areas with normal morphology in GEM-CaP mice. Foci displaying hyperplasia and/or nuclear atypia, also displayed Tag expression in the nuclei, indicating an association of SV40 Tag expression with cell proliferation and the onset of tumorigenesis. The extent of Tag staining increased significantly (P<0.01) from hyperplasia/WT to PIN till the WD and MD tumors (P<0.01), but it dramatically decreased (P<0.01) from MDCaP toward a conspicuous rare expression in a PD tumor.

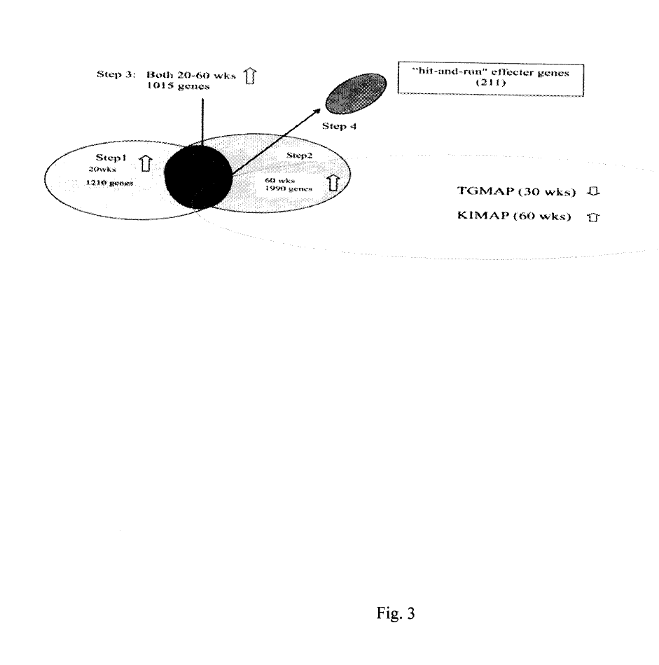

A differential cDNA microarray analysis was performed on two GEM-CaP models. Most of PSP94 transgenic mice (TGMAP) developed AI and NE carcinoma in lobes of the ventral prostate (VP) and dorsolateral prostate (DLP) lobes within 4 to 8 months of age, but as the KIMAP model showed a steady and synchronous tumor growth with the majority of well- to moderately-differentiated CaP at 20-60 weeks of age, the differential GeneChip screening was based on genes up-regulated only at the early stages of tumor development in KIMAP mice, and down-regulated at the later stage in the TGMAP model. Prostate tumor samples from transgenic models KIMAP mice from 20 weeks (n=7) and 60 weeks (n=10) were compared with wild-type in MOE430A GeneChip and KIMAP mice (60 weeks) and TGMAP (large tumor only, n=7) were compared in MG_U74Av2 GeneChip. For PSP94-TRIM59 mice, and also for additional GeneChip comparison between wild-type mice (n=12) and large sized TGMAP tumor tissues (n=7), all used the MOE430A GeneChip. In the first step of a four-step differential screening strategy (as shown in FIG. 3), 1210 genes were identified as the most up-regulated genes in KIMAP mice at 20 weeks of age, compared to wild-type prostate tissue, which represented tumorigenesis from PIN to WDCaP. In the second step, 1990 genes were further identified to be up-regulated at 60 weeks, which were mostly MDCaP. In the third step, among those 1990 genes, 1015 were up-regulated in both groups (20 and 60 weeks of age), indicating that they share similar functions. In the final step, from the 1015 genes, 211 genes were down-regulated in large-sized tumor bearing TGMAP mice, which were mostly at the late stages of AI (androgen-independent) or NE (neuroendocrine) carcinoma. Seventeen genes were excluded as they were duplicates. A total of 194 genes were analyzed as a global filing with functions correlating with SV40 Tag-induced tumorigenesis, which are involved in tumor initiation, but not tumor progression or metastasis. The results of functional analysis of these 194 genes according to NCBI and PubMed reveal that they are mostly (67%) CDC related genes: G1/S clusters, G2/M clusters, and M/G clusters are represented at 39.7%, 29.9% and 1.0% respectively. A high percentage of RNA processing genes (10.3%) and oncogene-related marker genes (7.7%) were also detected. Consistent with the Tag “hit-and-run” effect, the most SV40 Tag direct binding proteins and their down-stream effector genes, as well as CDC related genes were down-regulated in the late stages of tumor development (large sized TGMAP tumors.

Correlation of TRIM59 Gene Expression with the SV40 Tag-Mediated “Hit-and-Run” Effect in GEM-Cap Models:

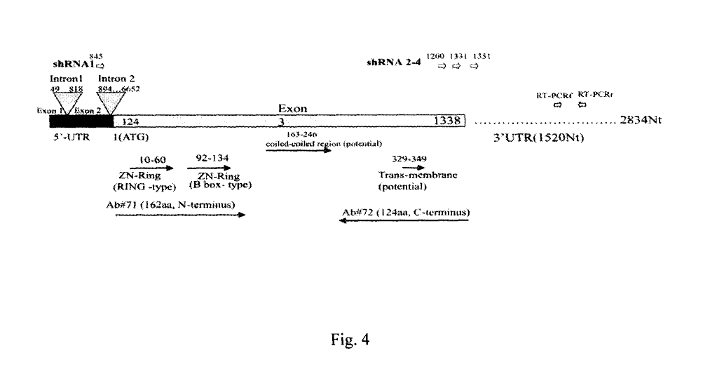

Based on differential gene chip profiling correlated with the SV40 Tag “hit-and-run” effect, TRIM 59 (NM_{—}025863) was selected, which was a hypothetical gene, for further studies. TRIM59 gene expression was up-regulated 16.84 and 24.07 fold at 20 and 60 weeks of age in KIMAP, respectively, compared to normal prostate tissue, while in TGMAP large tumors, TRIM59 was down-regulated. FIG. 4 shows a brief map of TRIM59 gene structure, cDNA and protein (ORF), as well as the locations of the hypothetical functional domains, and the extra-long 3′-UTR (1520/2834 nucleotides 1/2 of cDNA) of TRIM59 mRNA, which were obtained by searching the NCBI database.

To confirm the GeneChip results, a semi-quantitative RT-PCR determination of TRIM59 mRNA was performed. Primer pairs for RT-PCR were selected, close to the probes used for GeneChip analysis, and also located near the 3′-end of the cDNA sequence. Wild-type mouse prostate, cultured mouse fibroblast NIH 3T3 cells, and GAPDH were used as controls. Signals of RT-PCR fragment of TRIM59 were higher in GEM tumor at 20 weeks and 60 weeks of age, compared with controls of wild-type prostate tissue and NIH3T3 cells. To further test the presence, abundance, and size of the full length TRIM59 mRNA, Northern blots were prepared from total RNA preparations from NIH3T3 cells, KIMAP (20 and 60 weeks) and TGMAP (four late stage, large tumors) and hybridized with ³²P-dCTP-labeled 250 bp RT-PCR products. 2.5 kb mRNA was detected in GEM-CaP tissues, consistent with the predicted size by NCBI computational sequence analyses, whose levels were higher in KIMAP than TGMAP tumors.

To characterize the protein product of the hypothetical TRIM59 gene, two polyclonal antibodies were prepared against two GST-mouse TRIM59 fusion proteins separately. The first GST-TRIM59#U71 contains an N-terminal fragment (163 aa from cDNA sequence 127-616) covering several hypothetical BBRC domains. The second GST-TRIM59#U72 contains the C-terminal fragment (126 aa, cDNA sequence 961-1338) with no complete hypothetical functional domains determined by NCBI. The TRIM59#72 antibody was selected with the following characterizing results: (1) only one dominant band at 53 kDa was repeatedly observed by the TRIM-#72 antibody in protein samples from either cell lysates of NIH 3T3 cells, or CaP samples from KIMAP and TGMAP mice. The 53 kDa band is close to the predicted molecular weight (coding for 403 aa, 44.77 kDa, see FIG. 4) of the hypothetical ORF (cDNA 127-1338). The TRIM59-#71 antibody recognizes a 72 kDa band (major band) and a 65 kDa band, which are both higher than the expected molecular weight. The #71 antibody recognizes different phospho-(phosphorylated) proteins from elutions separating from an IMAC affinity column of total cellular p-proteins (see next section). The TRIM59#71 antibody only showed a very weak band at 55 kDa as a phospho-protein, which was shown as a major phospho-band by TRIM59#72 antibody. (2) both #71 and #72 recognized the same major band (53 kDa) in Western blots using a #72 affinity column purified TRIM59 protein (see next two sections). (3) As shown in FIG. 4, the TRIM59 C-terminal sequence contains less common functional domain, i.e. contains more TRIM59 specific sequence than the N-terminus. (4) By sequence search, TRIM59 contains no complete or part PRYSPRY sequence, which in some TRIMs is located at the C-terminal with IgG/Fc binding function.

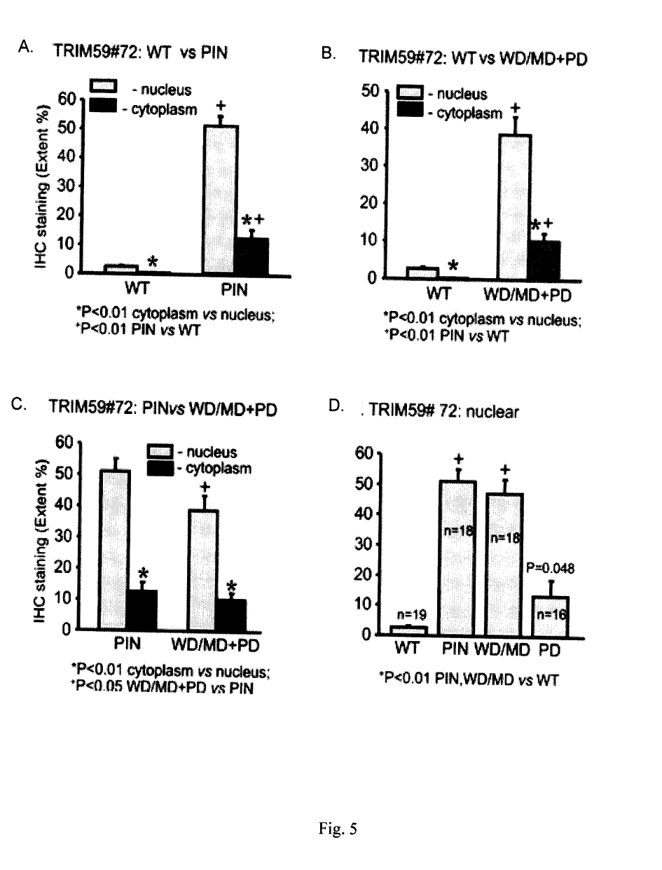

A systematic IHC analysis was then performed using TRIM59#72 antiserum in both KIMAP and TGMAP tumors. Foci were scored according to the extent of IHC staining by antibody TRIM59-#72, utilizing the same protocols as SV40 Tag staining. Representative IHC staining with 5 main grades of normal with hyperplasia, PIN, WD and MD and PD CaP were noted, while normal gland foci or wild-type mice showed no TRIM59 protein staining, consistent with SV40 Tag IHC staining. These results confirm the data from GeneChip and RT-PCR studies, and indicate that TRIM59 expression is also up-regulated in tumor tissues at the protein level. In addition, IHC staining for TRIM59 protein in PIN and cancer foci (WD/MD/PD) was found in both the cytoplasm and nucleus, while both were higher than the WT (P<0.01, FIGS. 5A and B). Nuclear staining in the cell proliferative area, i.e. PIN and in all cancer foci was significantly higher than in the cytoplasm (P<0.01, FIG. 5D), and higher than the staining of all cancer foci (P<0.01, WD/MD and PD CaP). However, there was no difference in cytoplasmic staining with cancer foci (FIG. 5C). Foci of WDCaP and MDCaP showed a similar extent of TRIM59 IHC staining. A close correlation of TRIM59 protein expression patterns and with the “hit-and-run” effects of SV40 Tag is demonstrated in FIG. 5D. In PDCaP, TRIM59 IHC staining signals (see FIG. 5D) were lower than those of PIN, WD, and MD CaP foci, especially when counting nuclear staining of TRIM59 (P<0.01).

To test if TRIM59 expression also correlates with tumor progression in human clinical samples, IHC analyses were performed as described on tumor biopsy samples from cancer patients from the prostate (n=4), bladder (n=7) and kidney (renal cell carcinoma, RCC, n=5). TRIM59 was shown to be up-regulated (3 of 5) in human kidney cancer (RCC).

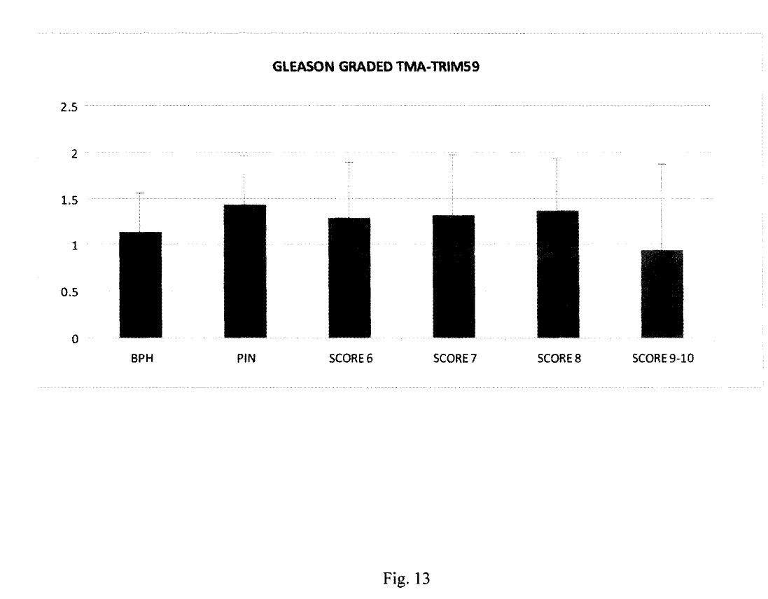

Human prostatectomy specimens were also analyzed using Gleason graded tissue microarray analysis. The results are tabulated below in Table 2 and illustrated in FIG. 13.

TABLE 2

| # OF CORES | GROUP | MEAN-INTENSITY | STDEV |
|---|---|---|---|
| 31 | BPH | 1.129032258 | 0.428 |
| 7 | PIN | 1.428571429 | 0.535 |
| 49 | SCORE 6 | 1.285714286 | 0.612 |
| 29 | SCORE 7 | 1.310344828 | 0.66 |
| 27 | SCORE 8 | 1.37037037 | 0.565 |
| 16 | SCORE 9-10 | 0.9375 | 0.938 |
| 5 | STROMA | 0.2 | 0.447 |
| 12 | ABSENT | N/A | N/A |
| | CORES | | |
| TOTAL | N/A | N/A | |

176

For BPH and PIN, TRIM59 was expressed in the baso-membrane of the basal and luminal cells. For Gleason 3+3, Gleason 4+4 and Gleason 5+5, TRIM59 was expressed in the cytoplasm of the tumor cells in various patterns, e.g. glandular pattern for Gleason 3+3, cribriform pattern in Gleason 4+4 and in Gleason 5+5, arranged in individual cells and tumoral sheets.

### Identification and Characterization of Phosphorylation Forms of TRIM59 Protein by IMAC Enrichment in Mouse Cell NIH 3T3 Culture:

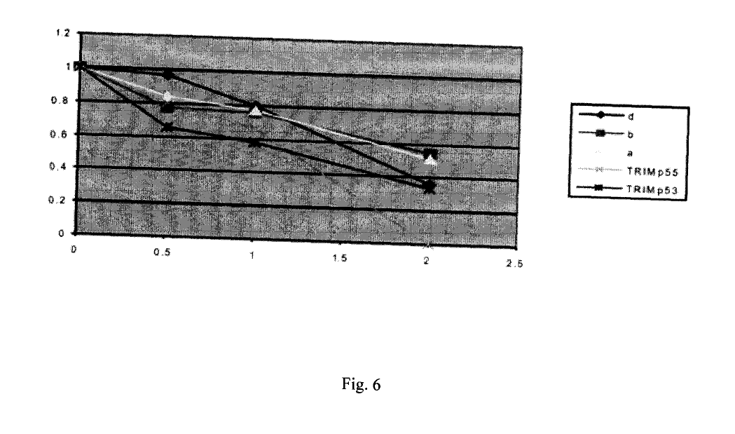

To demonstrate that TRIM59 expression is correlated with SV40 Tag induced tumorigenesis (“hit-and-run” effect), the state of phosphorylation of TRIM59 that correlates with the Tag tumorigenesis was characterized, since signal transduction of SV40 Tag effectors is linked with protein phosphorylation, and TRIM59 proteins are present in the nucleus in tumorigenesis and progression (FIG. 5D). An IMAC (Immobilized Metal Affinity Chromatography) enrichment of cellular total phosphorylated proteins in mouse fibroblast NIH3T3 cell lysates was performed. The intensity of a 53 kDa band in NIH3T3 cell lysates was not significantly changed after passing IMAC purification, indicating that the phosphorylated form may be a minor species. A careful quantization revealed that only approximately 1/250 of this protein was in a phospho-form (TRIM59-p53), along with another four phospho-proteins of 77, 62 and 40 kDa, respectively, which were all immunoreactive to TRIM59#72 antibody. As the TRIM59#72 antibody always detects only one major band (53 kDa) in Western blots in cell and tissue lysates, in order to differentiate these multiple #72 antibody positive phospho-proteins, phosphate (PBS) competition to IMAC purification was performed with gradient doses of PBS added in the cell lysates. Only two bands designated as TRIM59-p53 and -p55 were more sensitive to PBS competition (FIG. 6), and their molecular weights are closer to 53 kDa, which is the proposed non-phospho-form of TRIM59. These two forms of p-TRIM59 were identified with different IMAC columns (Qiagen and Clontech) and repeat purification procedures showed various forms of different relative intensity. To further confirm the IMAC results by Western blotting, recombinant GST-TRIM 59 were added as a competitor peptide of C-terminus antigen to the TRIM59#72 antibody reaction. The addition of increasing amounts of the C-terminal peptide significantly abolished the 53 kDa TRIM59 band. Immuno-precipitation by TRIM59#72 antibody immobilized to the gel matrix (Amino-Link, Pierce) of TRIM59 phospho-forms as demonstrated by ³²P labeling of total cellular phospho-components in cultured NIH 3T3 cells resulted in two major bands at apparent molecular weight of 72 and 53 kDa separately consistent with the IMAC results.

The same IMAC column enrichment was applied to GEM-tumor tissues. CaP samples were from both TGMAP (TG) mice (6 months of age in average, large sized PDCaP tumors, n=5) and KIMAP mice at different ages with WDCaP (20 weeks, n=12) to MDCaP tumors (40 weeks, n=11; 60 weeks, n=7). Each mouse was tested histologically and confirmed with the proposed tumor grade separately, then entered into this study. Following IMAC isolation of total tissue lysates for total phospho-proteins, two forms of TRIM 59 phospho-proteins, p53 and p55, were identified as the same molecular weights in the elution fractions as in that of NIH3T3 fibroblasts. TRIM59-p55 was found to be up-regulated in large sized tumors in TGMAP mice exclusively, and absent in all other tumors of KIMAP mice (20, 60 weeks). TRIM59-p55 protein appeared as a less focused band in Western blots, as compared with wild-type mice. The expression levels of TRIM59-p53 were lower in wild type prostate lysates and increased in samples from KIMAP age groups of 20 and 40 weeks, while the levels were lower in the 60 week groups and TGMAP mice. Two different internal reference controls were performed: (1) by antibody of β-actin for probing samples of tumor and tissue lysates normalized by wet weight and protein contents (50 μg/each). Since neither β-actin nor GAPDH can be used as references for total phospho-proteins, total protein content only was used as references, which correlated roughly with wet weights of tissues. (2) control experiments were conducted by passing through fractions using β-actin as the control.

To further ascertain TRIM59 phosphorylation, TRIM59 proteins were purified by affinity column purification and tested pooled lysates from large sized TGMAP tumors (n>10). Tumor lysates were loaded to the affinity column and the purified TRIM59 proteins were immuno-reacted with two kinds of antibodies against phospho-tyrosine (p-Y) and phospho-threonine (p-T, which most likely also stands for all p-S/T proteins) to determine the types of phosphorylation present. As a control, the same Western blots were probed for the second time using the TRIM59#72 antibody to identify total TRIM59 proteins. A band of 53 kDa was repeatedly observed at the same molecular weight as the product of the IMAC column. Phospho-TRIM59 with tyrosine residues (p-Y-TRIM59) showed slightly higher mobility in SDS-PAGE than the non-phospho-form. Phospho-TRIM59 with threonine residues (p-T-TRIM59) showed approximately the same mobility in SDS-PAGE as the non-phospho-form. Densitometry scan (data not shown) showed that approximately 30% and 70% of total TRIM59 proteins were p-Y and p-S/T residues, respectively.

To confirm the specificity of the re-probing with the p-tyrosine antibody, an immobilized gel matrix conjugated with the p-Y antibody was utilized to immuno-precipitate the products purified by the TRIM59#72 affinity column. Only one 55 kDa band was detected by the p-Y antibody in Western blots.

To further confirm the results from the affinity column purification, the same tumor lysates were used for purification of TRIM59 proteins by IP (immunoprecipitation) with TRIM59 antibody immobilized on agarose gel matrix. Since the molecular weight of TRIM59 proteins is close to that of the heavy chain of IgG, we tested each time of IP reactions for pretreatment washing (by elution, binding, and washing buffer) to confirm the complete coupling and the minimized leaking levels of antibody from immobilized gel. A major band of 53 kDa TRIM59 protein was confirmed in the elution fraction. Two parallel Western blots were immuno-reacted with two kinds of antibodies against p-T (Ab-p-T) and p-Y(mAb-p-Y) proteins. Both of these two antibodies detected positive signals in the elutions. Stronger signals by Ab-p-T were found in elutions than in those of mAb-p-Y. The yield of TRIM59 phosphoproteins was measured as only 30% of that by the TRIM59 affinity column as a semi-quantitatively comparison from gel densitometry scanning. However, the p-Y signal was significantly lower than those of p-T, similar to the results from the affinity column.

### Characterization of Phospho-TRIM59 Forms by Western Blotting and ELISA in Different Grades of the GEM-Cap Tumor:

A semi-quantitative comparison of the two forms (p-53 and p-55) of total TRIM59 proteins was then performed. All TRIM59 proteins tested were from affinity column purification from NIH 3T3 cells (1×10⁸ cells), from pooled tumor samples from KIMAP (n=15, 20-40 weeks of age) and from TGMAP (large-sized, later stage CaP, n=5) mice. To normalize the total TRIM59 protein loaded, all Western blots for semi-quantitative comparison were loaded in parallel with the affinity column purified TRIM59 proteins from TGMAP tumors (TG), and first tested by TRIM59#72 antibody. Elution fractions from NIH 3T3 cell lysates revealed the same 53 kDa band as in the large-sized TGMAP (TG) tumors. After normalization with TGMAP tumor tissues, two identical Western blots were prepared and loaded with the same amounts of total TRIM59 purified proteins from NIH3T3 and TGMAP tumors. Two lanes of TG samples were loaded comparable concentrations (TG2×1, TG×5). These two blots were probed with two specific antibodies against p-T and p-Y separately. TGMAP large-sized tumors showed higher p-Y phosphorylation band than that of NIH 3T3, while NIH 3T3 still revealed certain levels of p-T phosphorylation, although lower than that of TGMAP. Similar comparison by semi-quantitative Western blots was performed for affinity column purified TRIM59 samples from KIMAP tumors along with TGMAP samples. Normalization was performed by testing with TRIM59#72 antibody and followed by two identical blots with two antibodies, p-T and p-Y. Results of the comparison showed that in both KIMAP and TGMAP tumors, there were high levels of p-S/T phosphorylation. TGMAP large-sized tumors showed the highest level of p-Y phosphorylation.

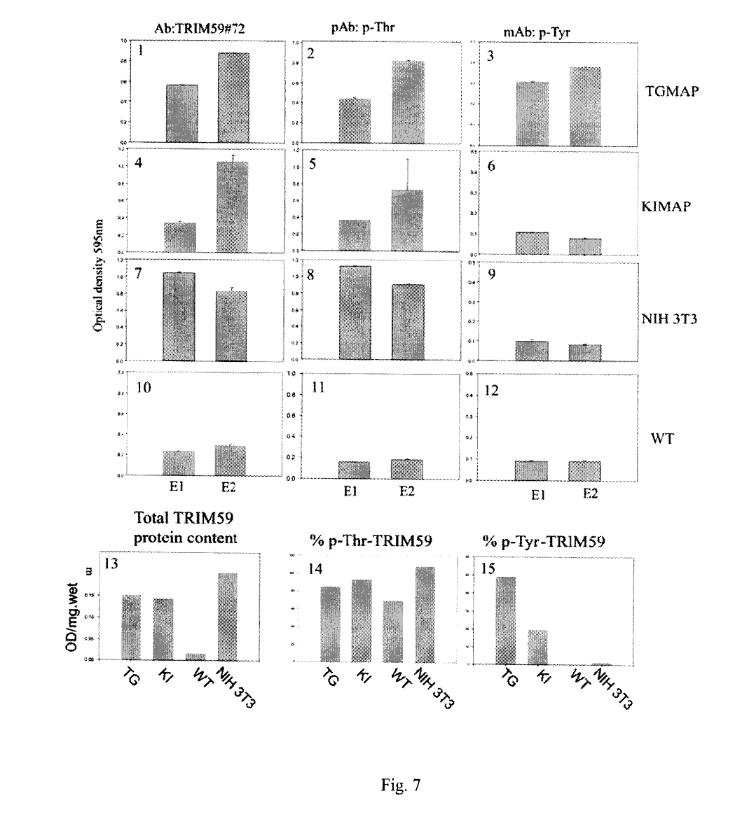

Since results from semi-quantitative Western blotting analyses clearly indicate the specificity of the TRIM59#72 antibody and two other anti-phosphoprotein (p-Y, p-T) antibodies to test affinity column purified proteins, an ELISA protocol was established to precisely quantify the levels of purified TRIM59, to examine the correlation between TRIM59 and p-TRIM59 and tumorigenesis and tumor progression. To this effect, prostate tissue samples from KIMAP (n=10, 20-40 weeks of age with well to moderately differentiated tumor) from 5 large-sized TGMAP tumors was examined, together with two controls from pooled VP (ventral) and DLP (dorsolateral) prostate tissue samples from 20 wild-type mice (WT age matched, 6 months old) and NIH 3T3 cells (1×10⁸). The first two elution fractions (E1 and E2) were tested separately as it was found by Western blotting that the TRIM59#72 affinity column can separate two forms of p-TRIM59 proteins in these two fractions. TRIM59 elution proteins (E1, E2) were coated in duplicate or triplicate and first tested with increasing amounts by ELISA with TRIM59#72 antibody. This result served as reference to normalize all samples tested at OD₅₉₅ₙₘ (optical density of 595 nm) ranging from 0.5-1. FIG. 7 includes graphs that show results of ELISA quantization of three identical sets of samples of purified TRIM59 proteins tested in parallel with three antibodies against TRIM59#72, p-S/T (polyclonal, pAb-p-Thr) and p-Y (monoclonal mAb). Graphs 14-15 in FIG. 7 summarize ELISA results. (1) TGMAP mice (graphs 1-3, FIG. 7) have the highest (2-3 times) levels of p-Y phosphorylation than all other samples (KI, WT and NIH3T3, graphs 3,6,9,12, FIG. 7) (2) All GEM-CaP mice (TG, KI of graph 2, 5 of FIG. 7) had higher p-Y phosphorylation than wild-type mice (graphs 2,5,11,14, FIG. 7), as well as higher levels of total TRIM59 protein levels (graphs 1,4,10,13 of FIG. 7). (3) NIH 3T3 displayed higher p-S/T levels than all GEM-CaP mice (FIG. 7 graph 2,5, 14 vs 8), but the same low levels of p-Y phosphorylation as KIMAP (FIG. 7, graph 6,9,15). Wild-type mice had the lowest levels of total and phosphorylated TRIM59 (FIG. 7, graph 10-12 and 13). TGMAP mice showed higher total TRIM59 protein than KIMAP mice, which may be due to more proteins being diffused from nuclear (KIMAP) into cytoplasm (TGMAP) as indicated by IHC studies. Elution fractions of E1 and E2 from KIMAP tumors showed separation of p-S/T (E2) and p-Y (E1).

TRIM59 mRNA Knockdown in Human Prostate Cancer Cells Results in S-Phase Arrest and Cell Growth Retardation Along with a “Hit-and-Run” Effect in the K-Ras Signalling Pathway:

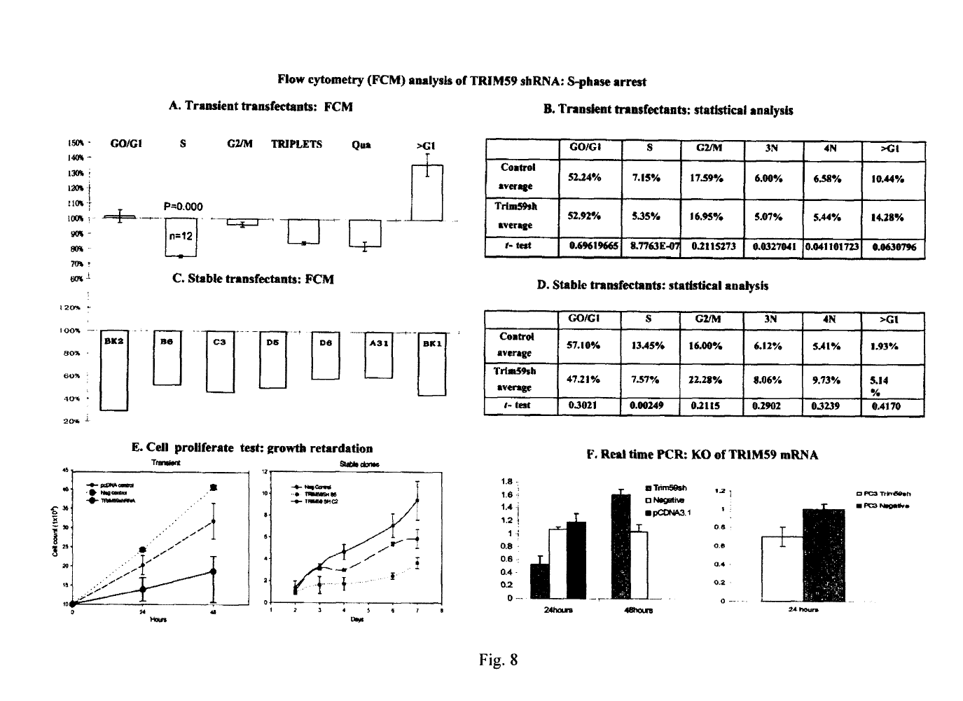

To investigate the mechanism of TRIM59 function, its expression was reduced by knockdown (KO) of TRIM59 gene expression, using four shRNA plasmid constructs, namely, 5′ end (sh1) and 3′ end (sh2, sh3) of the human TRIM59 ORF, and the 3′UTR (sh4), close to targets of many microRNAs including miR17 (FIG. 4). Transient transfection (FIG. 8A) of pooled plasmid DNA of these four shRNA's into the human prostate cancer cell line DU145, revealed a statistically significant (P=8×10⁻⁷, FIG. 8B) decrease in the population (%) of S— phase cells by flow cytometry (FCM), compared with cells infected with the control pSilencer vector (a shRNA vector with non-specific insert from Ambion. This result was highly reproducible (n=20) in both DU145 and PC3 cells (data not shown). Stable transfectant clones (Neo^{R} n=7) in DU145 cells also demonstrated similar results (FIG. 8C/D), i.e. S-phase cells from G0/G1 in FCM had significantly (P=0.002) lower TRIM59 levels than all other cell groups. Up-regulation of the TRIM59 gene in DU145 cell did not show FCM abnormality or aneuploidy changes (data not shown). Cell proliferation tests were performed by cell counting at different time-points (FIG. 8E). Both transient and stable transfectants showed that TRIM59 KO resulted in a significant retardation of growth of the human prostate cancer line DU145 (50-30% of the control group). The degree of down-regulation of TRIM59 mRNA was determined by real time RT-PCR. FIG. 8F shows that only after 24 hours of transient transfection in DU145 and PC3 cells, TRIM59 mRNA was decreased by 50% (n=4) as compared with the negative shRNA plasmid controls, as well as pcDNA plasmid (FIG. 8F). The TRIM59 mRNA levels returned to normal 48 hours after transfection (FIG. 8F), in all stable transfectant clones selected, in both the DU145 and PC3 cell systems.

TRIM59 protein levels were tested by semi-quantitative Western blotting experiments. As compared with HEK293 cells, all human prostate cancer cells from strains of LNCaP, DU145, and PC3 showed higher levels of TRIM59 protein (53 kDa). In contrast, shRNA transfectants showed lower TRIM59 protein levels as compared with controls and the internal reference of β-actin. To test the TRIM59 protein phosphorylation levels in shRNA KO transfectants, IP of TRIM59 in stable transfectant cells was performed. Results showed that the phosphorylation levels of both p-S/T and p-Y were also decreased to a level corresponding with the decreased TRIM59 protein in shRNA transfectants.

To study the possible targets of TRIM59 function after shRNA KO, gene profiling of stable transfectant clones (S) was analyzed with the negative plasmid as a control for normalized expression test. A chip heatmap of “S” clones of gene profiling with a 2-fold decreased gene expression (n=148 including repeated genes) was generated with groups classified according to their reported functions. The first group was protein phosphorylation in MAPK/ERK and PI3K/Akt pathway (n=13), which may account for the decreased TRIM59 phosphorylation. The second group is composed of decreased expressed genes related to cell proliferation and CDC (cell cycle division) regulation (n=4) and S-Phase (n=3), which may account for effects of S-phase arrest and growth retardation. The third groups contain several signal pathways including the Rho GTPase family (n=4), G-protein (n=3), NF-κB (n=2), ETS (n=1), Wnt and catenin (n=4), BMP4-Smad pathway (n=1) and insulin-like growth factor family (n=2), including group related to P53 and chromosome stability (n=5). Decreased gene groups of “S” clones are also from genes related to ion channels (n=5), cytoskeleton (n=9) and chaperone/stress responsive genes (n=8), which may reflect the remodeling of cytoskeleton resulting from the effects of agonist-receptor binding in Ras signal transduction, small GTPases, Rho signaling, and PI3-kinase. Other effects of TRIM59 KO involve the decreased expression of genes related to NE (Neuroendocrine) carcinoma (n=3), and immuno-response (n=2), which indicates TRIM59 gene function in tumor progression. “S” Chip also is comprised of a large proportion of genes with decreased expression of unknown function or the mechanism not well studied: “others” (n=11, function not related with cancer), tumor suppressors and tumor markers (n=18, function only listed as tumor marker) and hypothetical genes (n=30, no research reports in PubMed). Gene-up profiling of “S” clones was also analyzed, which are for the most part related to immuno-response genes (CD24, chemokine interferon, etc), possible antagonist genes of the “decrease” lists.

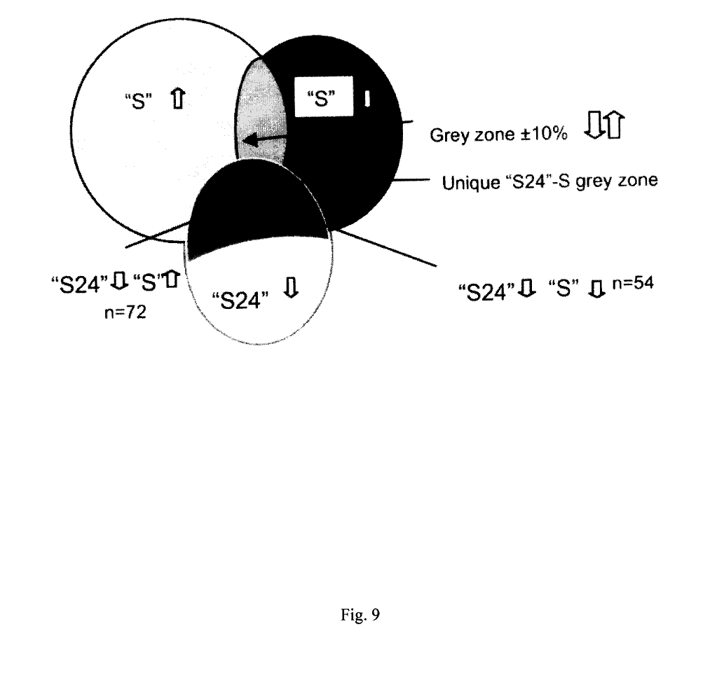

Due to a potential “hit-and-run” effect of the signal transduction, only in transient transfection can the original signal transduction targets be detected by comparison of gene profiling, since in stable shRNA transfectant clones, TRIM59 mRNA levels, although showing the same phenotype as the transient 24 hours transfection (“S24”), remain unchanged (FIG. 8F). When normalized by negative shRNA control plasmid GeneChip signals, “S24” was low in the 1.5 fold decreased gene list (n=20), which may be due to the limit of transfection efficiency and the short expression time of shRNA plasmid expression. TRIM59 mRNA in“S24” was 16% lower than that of “S” shown in GeneChip assay. Two kinds of differential Chip analyses were performed (shown in FIG. 9) based on gene profiling uniquely displayed in a 24-hour transient transfection sample (“S24”). The first was the Unique “S24”−“S” grey zone” (n=43), by screening the listed “S24”genes that were uniquely decreased 1.5-fold while “S” was unchanged (“grey zone”). The second was Unique “S24“−”S”D (n=59), which was a similar listing with decreases (>1.3 fold) between the “S24” and the “S” genes (decreased or increased). In both “Unique S24-S grey zone” and “Unique “S24”−“S″D” maps, differential GeneChip lists show a unique and fast decrease of genes in the K-Ras pathway corresponding to the TRIM59 gene KO: KRas (v-Ki-Ras2 Kirsten rat sarcoma viral oncogene homolog), RasSF5 (Ras association RalGDS/AF-6 domain family 5), FGFR1, and FGF14 (none are typical FGF family members), together with several phosphorylation genes. Wnt/Notch/catenin pathways were also targeted as early decreased expressed genes. Changes in S-phase and cell proliferation related genes in “Unique S24” may account for the S-phase and cell growth retardation 24 hours after TRIM59 shRNA. In both “UniqueS24” lists, almost half the genes were not well studied (“others”) or were just hypothetical genes, indicating TRIM59 function as a novel signal pathway.

Transgenic Mouse Modeling of PSP94-TRIM59 Up-Regulation Demonstrates TRIM59 Gene as a Proto-Oncogene in Tumorigenesis and NE Tumor Progression with a Gene Profiling Bridging Between SV40 Tag and Ras Signal Transduction Pathways:

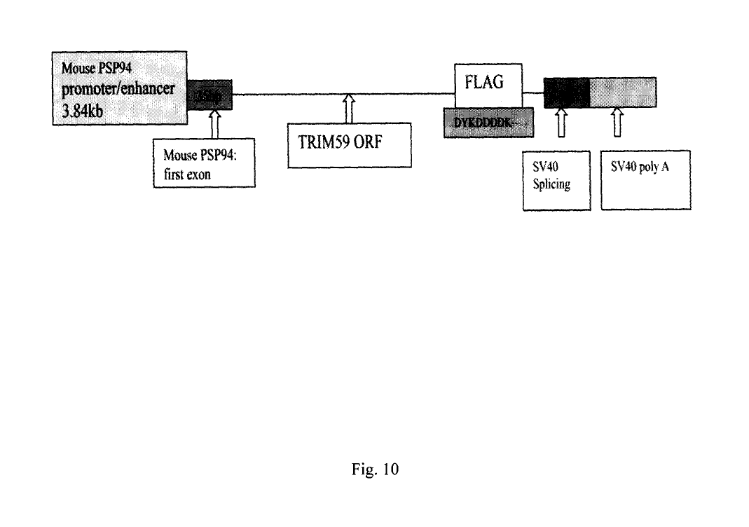

To test the potential of the TRIM59 gene as a novel proto-oncogene (i.e. the up-regulation of a single TRIM59 gene) that can induce tumorigenesis as in the SV40Tag oncogene in mouse GEM-CaP models, the prostate-specific gene promoter of the PSP94 gene was used to direct up-regulation of mouse TRIM59 gene expression in a transgenic mouse test. The transgene structure is shown in FIG. 10, in which the TRIM59 ORF was modified by insertion of a FLAG (DYKDDDDK), an immuno-epitope tag, and followed by SV40 small-t splicing and poly A tail sequences. Four F0 breeding lines (F3 with 60 male mice) were established. At first, 15 mice aged 100-110 days from four breeding lines were first dissected for histopathological analysis. H&E staining of formalin-fixed sections of prostate samples (VP and DLP lobes) showed that 3 mice developed WDCaP mostly in DLP, 6 mice had developed low to high grade PIN and 6 mice showed normal prostate tissue (except for some hyperplasia). PSP-TRIM59 mice exhibited PIN structures demonstrating multiple layers of epithelia gland, with deep nuclear chromatin staining, existence of multiple nucleoli with enlarged nuclear size, and increased numbers of mitotic cells depicting higher cell proliferation. Atrophic glands were often observed. PSP94-TRIM59 mice at 110 days also showed invasion of the surrounding glands and formation of fused glands, indicating early tumor progression. Some apoptotic bodies were observed, although less often than TGMAP or KIMAP tumors. Moderately differentiated tumor was observed (170-200 days of age, n=3) with the formation of multiple small grand, and fused glands, the glandular differentiation being similar to the knock-in of SV40Tag (KIMAP) mice and in human CaP. A PSP-TRIM59 mouse with poorly differentiated CaP was also observed. A highly invasive CaP structure of comedo-carcinoma and comedo-necrosis was seen, which shows typical features of NE (small cell carcinoma) and AI CaP. In a total of 26 PSP94-TRIM59 mice analyzed, 6 mice (23%) had cancer mostly in DLP (the prostate lobes most sensitive to carcinogenesis in rodents) with WDCaP, while 15 (57.7%) of the mice showed normal structure.

The PSP94-TRIM59 transgenic expression was confirmed by RT-PCR utilizing primer pairs of FLAG (3-end) and a 300 bp upstream oligonucleotide, with the wild-type mouse as the control. Higher TRIM59 expression was found mostly in PIN and WDCaP foci, as compared with the normal gland. IHC staining signals of TRIM59 protein was mostly in the cytoplasm and only 10% in nucleus, as measured by cells (323:30) in each slide view. An in vivo cell proliferation test was performed and results showed that in the PSP94-TRIM59 mice, uptake and incorporation of BrdU occurred in foci having gland proliferation, in PIN and in WDCaP areas, which was higher than normal, and was also found in some of the KIMAP glands.

TRIM59 proteins purified by affinity column from the pooled prostate samples from PSP-TRIM59 mice (n=7) were also studied. The results of semi-quantitative Western blotting showed that TRIM59 protein levels, p-S/T phosphorylation forms of TRIM59 in PSP-TRIM59 mice were higher than the wild-type control. Both p-Y-TRIM59 were at very low levels.

To further characterize the oncogenic nature of PSP94-TRIM59 mice, GeneChip analysis was performed. 10-fold up-regulated genes were first analyzed and the majority of 10-fold up-regulated genes were found to be tumor markers (n=32, 23.8%), including tumor marker genes with functions not well studied (36/26.9%), confirming the PSP-TRIM59 model as a CaP model. In PSP94-TRIM59 up-regulation model, it was also verified whether or not the initial targets of the TRIM59 gene function are in Ras signal pathway as indicated in shRNA KO (“UniqueS24”) of TRIM59 in cultured human CaP cell lines. The 10-fold up-regulated genes related to Ras signal pathway are all Ras related adapter/chaperonin proteins in the cell membrane, for example, the 14-3-3 family member of Pla2g2a (16.39-fold up-regulated, phospholipase A2, group IIA), Styk1 (12.13-fold up-regulated, S/T/Y kinase 1) are related to Raf activation. 66 up-regulation genes with 2-10 fold are from the Ras pathway. As the Ras activator, Rho factors and G proteins (coupled receptors) comprises a large proportion (38.4%, 24/66) in the list of 3-fold up-regulated Ras related genes. TRIM59 gene up-regulation (2.24-fold) was also with all 2-fold up genes in PSP-TRIM59 mice. Most of Ras related genes up-regulated in PSP-TRIM59 model are not up-regulated in SV40 Tag derived KIMAP model, except for a few genes, such as (KIMAP/PSP-TRIM fold): Fos (FBJ osteosarcoma oncogene, 9.282/6.77) Shc (SH2-domain binding protein 1, 2.665/2.571), Rac)GTPase-activating protein 1, 2.571/4.988), Junb (Jun-B oncogene, 2.169/1.749), G protein-coupled receptor 125 (2.109/0.599). Most of SV40Tag binding/effector genes (RB and p53) including E2F family up-regulated in KIMAP models were not up-regulated in either 10-fold or 2-fold up gene lists in the PSP-TRIM59 model, except for a few genes, such as Rbbp4 (Retinoblastoma binding protein 4), Rbbp8 (Rbbp8 Retinoblastoma binding protein 8), Centromere protein E (CenpE), MAPK11 (mitogen-activated protein kinase 11), Ccnb2 (Cyclin B2), Cell division cycle 25 homolog B, and pRB effector E2f2 (E2F transcription factor 2). The exceptional genes in these comparison tables are possibly linkage genes between Ras, TRIM59, and SV40Tag pathways.

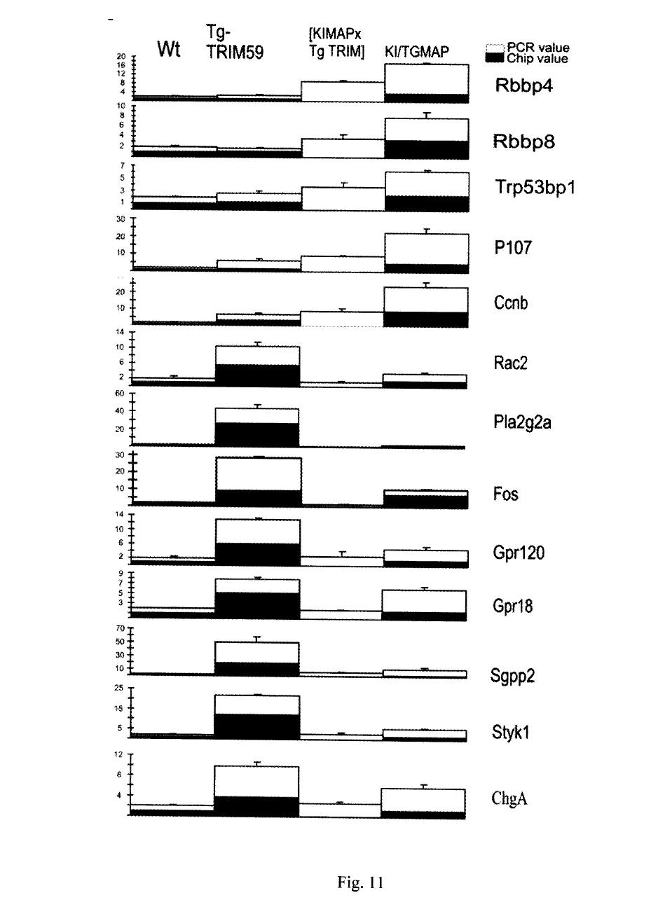

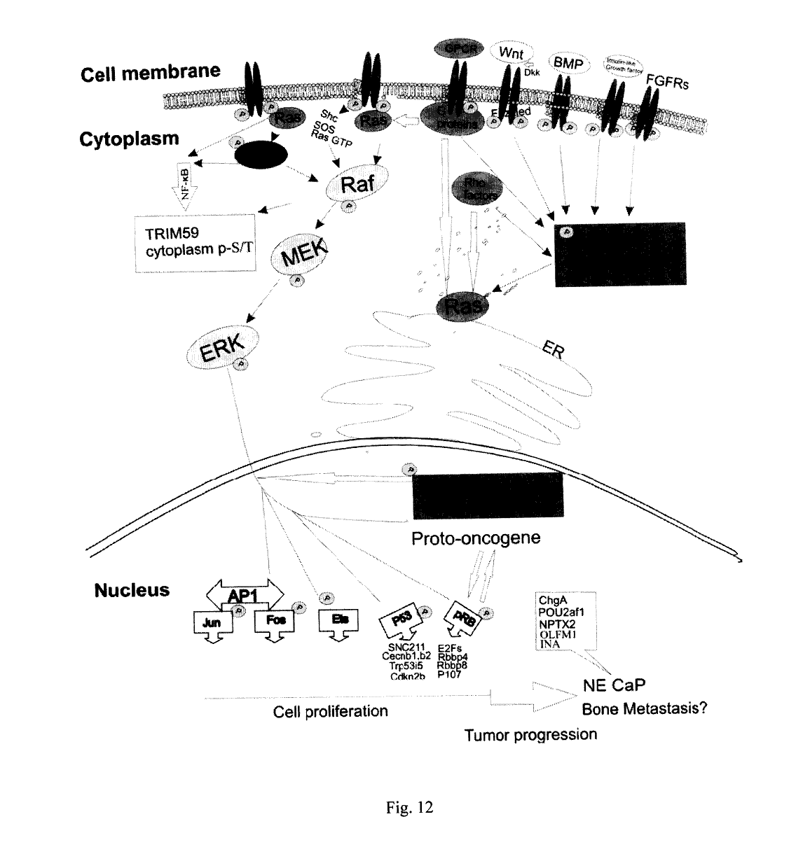

To verify the GeneChip results, real-time RT-PCR was performed on Ras, RB-p53 and NE-CaP related genes on RNA preparations from prostate tissue samples from Wt, Tg-TRIM59, KIMAP and hybrid of F1 (PSP-TRIM59×KIMAP) mice. FIG. 11 shows the results of the real-time RT-PCR determination of seven Ras related genes (Rac2, Pla2g2a, Fos, Gpr120, Gpr18, Sgpp2, Styk1), four SV40Tag effector genes (Rbbp4, Rbbp8,Trp53 bp1, Ccnb1-rs1, P107) and one NE-CaP marker (ChgA, Chromogranin). Real-time PCR results confirmed: (1) Ras related genes (Rac2, Gpr120, Gprl 8, Pla2g2a, Sgpp2, Styk1) are up in tg-TRIM59 and higher than that in KIMAP or hybrids of tg-TRIM×KIMAP mice; (2) All SV40 Tag effectors are higher than either tg-TRIM59 or hybrids of tg-TRIM59×KIMAP. In tg-TRIM59 and hybrids of tg-TRIM59×KIMAP, Rbbp8, Trp53 are higher than others tested, which confirms supposed functions as bridging genes between Ras and pRB. The low level of those bridging gene expression may be explained by the fact that as low as 16% of TRIM59 gene expression in “S24” in 24 hours is enough to ignite pathway signals for both S-phase arrest and growth retardation. (3) In hybrids of F1 [tg-TRIM×KIMAP], SV40 effector genes (pRb/p53) related genes are higher, although still lower than KI/TGMap models, than Ras related genes, indicating a dominant role over Ras related genes. In KI/TGMAP and in hybrids, Ras related genes (e.g. G-proteins) still detected with expression, indicating the linkage of SV40 Tag (pRB/p53) and Ras signal pathway. (4) NE-CaP marker of chromogranin A is significantly higher in tg-TRIM59 than KI/TGMAP and their hybrids.

### Discussion

The function of a novel TRIM family member, TRIM59, in SV40 Tag-directed transgenic and knock-in mouse CaP models is herein elucidated and characterized. By a systematic differential GeneChip screening approach, the TRIM59 gene was identified to be significantly correlated with the SV40 Tag “hit-and-run” effect, which SV40 Tag oncogenesis starts in cell proliferate PIN foci, but is down-regulated in the late stages of cancer, indicating a role in tumorigenesis.

As a down-stream effecter of SV40Tag in the tumorigenesis signal pathway, TRIM59 is regulated via post-translational phosphorylation. TRIM59 protein hyper-phosphorylation was evidenced using phosphoprotein IMAC affinity column enrichment, from in vivo labeling by ³²P—[H₃PO₄] and from the characterization of two phosphorylated forms of purified TRIM59 proteins by both affinity column and immunoprecipitation, using two kinds of phosphorylated protein specific antibodies. TRIM59 hyper-phosphorylation correlates with SV40 Tag oncogenesis. As demonstrated by both Western and ELISA experiments, TRIM59 hyper-phosphorylation on p-S/T TRIM59 is detected when SV40 Tag initiates tumorigenesis, and is then maintained at a relatively stable level during further tumor progression. P-Y TRIM59 is associated with advanced prostate cancer, the AI and NE CaP stage. TRIM59 gene expression, as compared with wildtype mouse, is up-regulated at the early stage when tumor is initiated in an SV40 Tag derived transgenic and knock-in mouse models. This up-regulation was also synchronously correlated with SV40 Tag oncogene expression, which may associate with the p-S/T phosphorylation of TRIM59. The down-regulation of TRIM59 gene expression is replaced by hyper-phosphorylation (by p-Y) in tumor late stages of the AI and NE carcinoma in SV40 Tag oncogene GEM-CaP mice. TRIM59-p55 is p-Tyr-TRIM59 and TRIM59-p53 is p-S/T-TRIM59, since in Western blots using specific antibodies against p-Y, and p-S/T protein, that only in large-sized TGMAP tumors, purified TRIM59 proteins either by affinity column or immobilized immunoprecipitation, showed high levels of p-Y-TRIM59 proteins.

As with all SV40Tag effectors, TRIM59 function also is involved in CDC regulation. By shRNA knockdown of TRIM59 in transfectant DU145 cells, TRIM59 is shown to play an important function in S-phase and cell proliferation, since KO of TRIM59 mRNA by 30-50% is enough to arrest cell in completing S-phase DNA duplication and also significantly retards cell division and growth even after 24 hours of shRNA KO. This function has been confirmed in a PSP-TRIM59 transgenic mouse model for testing the effect of up-regulation of TRIM59 gene specifically in the prostate, which as tested by BrdU in vivo labeling, accelerated cell proliferation and was one of the factors inducing CaP. The functional connection of TRIM59 in S-phase is closely related to the pRB/E2F route.

Although TRIM59 was screened and demonstrated to be an effector of the SV40Tag/pRB oncogenesis signal pathway, it was found that the initial functional targets of TRIM59 are actually in the Ras oncogene signal pathway. This was demonstrated by a differential GeneChip characterization of transfection of shRNA KO of TRIM59 gene comparing 24 hour transient and 48 hour until stable KO. In the “Unique S24” GeneChip list, there are almost no genes downstream of Ras that activated and induced the signal transduction cascade, including the mitogen-activated protein kinase/ERK kinase (MEK), extracellular-signal-regulated kinase (ERK), the PI3Ks (phosphatidylinositol 3-kinases), the RAL-activating RALGDS proteins ribosomal S6 kinase (RSK) and their down stream nucleus transcription factors NF-κB/CREB/ETS/AP-1 (Jun, Fos) and c-Myc. There were also no list of Raf up-regulated, down-stream cyclins such as cyclin D1, cyclin E, Cdk2, and Cdk4 etc.

This result was also confirmed in the PSP94-TRIM59 up-regulation transgenic mouse model, in which genes related to Ras signal pathway are the most significant up-regulated group. Activators of the Ras GTP/GDP cycle, RHO and G proteins coupled receptors, are also significantly up-regulated, which includes the majority of the up-regulated gene list. The same GeneChip analysis of PSP94-SV40Tag directed GEM-CaP models contained mostly pRB and p53 initiated signal transduction, which resulted in an abnormality of the CDC checkpoint system and chromosome instability.

Ras proteins bind to and activate the S/T kinase Raf, which then initiates a signal transduction cascade. Cytoplasm Raf is a pS/T kinase. This T/S phosphorylation is co-incident with TRIM59 protein phosphorylation as characterized in the GEM-CaP models, indicating the link of Tag/RB/P53 and Ras/Raf signal pathways. MEK is a Y- and S/T-dual specificity protein kinase. This is down stream of activated Rafs and induces a signal transduction cascade, which is also correlated with the p-Y phosphorylation of TRIM59 in late stage PSP-Tag models. All these Ras down stream effectors also down regulated or eliminated in shRNA down-regulation and KO of TRIM59 as shown in “unique S24” gene list, including those cyclins expression normally Ras up-regulates.

TRIM59 significantly revealed full potential as a proto-oncogene in transgenic mice, which is co-incident with up-regulation of TRIM59 in the prostate at both mRNA and protein levels. The oncogenic nature of PSP94-TRIM59 model has been characterized, which include histo-pathological gradings displaying a complete process of tumorigenesis and progress, and particularly a poorly differentiate comedo-carcinoma structure.

One feature observed in the PSP-TRIM59 model is the NE CaP (the Comedocarcinoma) differentiation observed. Results of the two GeneChip analyses on targets of shRNA KO and the PSP-TRIM59 transgenic model all indicate that neuroendocrine-related genes may be involved in TRIM59 signal transduction. For example, important NE CaP markers of POU domain associate factor, chromogranin A increased 28 and 3.69 fold separately. The targets of NE related genes also involved in some ion channel changes, as in TGMAP. This indicates a novel route implicating the linkage of SV40 Tag and NE carcinoma tumor progression through TRIM59 up-regulation and hyper-phosphorylation. TRIM59 also appears to be involved in the initiation of bone metastasis related genes, as BMP/SMAD pathways were the targets of “S” and PSP94-TRIM59 transgene, which may be accelerated by Wnt, NF-κB and PI3k/Akt pathways.

Ras resides near the cell membrane and pRB is a nucleus protein, which regulates proliferation and differentiation. In the SV40Tag derived GEM-CaP models, TRIM59 expression was mostly observed in the cytoplasm, which is different from SV40Tag and all other Tag binding proteins/effectors. TRIM59 expression actually correlated with Ras signal transduction from cytoplasm to nucleus. This report demonstrates that in spite of their geographical distance, intimate communication takes place between Ras and pRB, through various signaling channels.

The association of TRIM59 function with Ras signal pathway should not be considered to have any connection with the SV40 Tag/pRB/p53 tumorigenesis route, or as a single Ras/TRIM59 pathway induced tumorigenesis. This is because the TRIM59 function as a proto-oncogene is characterized as a signal pathway network in a powerful promoter of PSP94 directed SV40Tag oncogene initiated GEM-CaP models. Actual levels of TRIM59 protein was quite abundant in cell culture and mouse tissues. The activating TRIM59 in the nucleus may be from cell cycle cdk system coincident with activation of Tag/Rb/p53 pathways.

Thus, in conclusion, a new signal pathway bridging k-Ras and Rb signal pathways has been identified which is mediated through TRIM 59 function in tumorigenesis and progression, since the TRIM59 gene is a novel computer-predicted gene and a novel effector of SV40Tag tumorigenesis signal pathway, and TRIM59 is also a novel proto-oncogene in Ras signal pathway, as characterized herein. It appears that this new signal transduction route, which shows two forms of TRIM59: TRIM59-cytoplasm (p-S/T) and TRIM59-nucleus (p-Y), is present in Ras and Rb related signal pathways, separately. TRIM59-cytoplasm linked with Ras family members is regulated with several RHO and G-protein members, and shows the cross linkage of multiple cellular membrane bound pathways: Wnt-β-catenin, BMP-SMAD, insulin like growth factors, FGFR, etc. pS/T TRIM59 phosphorylation appears to correlate with Ras/RAF activation in the cytoplasm and p-Y-TRIM59 hyper-phosphorylation is associated with MEK/ERK/P133K/AKT phosphorylation as well as CDK systems in the nucleus and the sequential activation of other oncogenesis in the nucleus. The novel TRIM59 signal pathway explained an oncogene potentially directing tumor progression to NE and bone metastasis in SV40Tag directed GEM-CaP models, which also resulted in hyper-phosphorylation of p-Y-TRIM59 in nucleus.

Thus, it has been found that a novel TRIM59 gene as a proto-oncogene can affect both Ras and RB (SV40 Tag oncogene target) signal pathways just by up/down-regulation its function in DNA synthesis (S-phase). These findings provide novel methods of diagnosis, prognosis, and therapy of cancer.

### Example 2

### Methods and Materials

All patient samples were acquired as part of REB (Research Ethics Board) approved protocols at the University of Western Ontario (UWO) and Vancouver Prostate Center, University of British Columbia (UBC). Table 1 (set out in the Results section) shows a complete list of 289 patients with 37 different tumor types examined in this study.

Prostate cancer Tissue Microarrays (TMA): 88 CaP patients between 2006 and 2008 who had no treatment prior to radical prostatectomy, were selected from the Vancouver General Hospital as described in Example 1.

Automated image, acquisition and analysis on immunohistochemical staining of CaP-TMA: (UBC) Immunohistochemical staining was conducted by Ventana autostainer model Discover XT™ (Ventana Medical System, Tuscan, Ariz.) with enzyme labeled biotin streptavidin system and solvent resistant DAB Map kit. TMA was scanned by Bliss Digital imaging system using x20 objective, from Bacus Laboratories INC, Centre Valley Pa., and stored in the Prostate Centre Saver (http//bliss.prostatecentre.com). A value on a four-point scale assigned to each core.

Multiple-tumor Tissue Microarray construction: Tissue samples form 42 patients that encompassed 35 distinct tumor subtypes were selected from London Laboratory Service Group, and the tumor bank in the Department of Pathology (UWO, by pathologist Dr. M Moussa). TMA slides were constructed with triplicate cores for each sample following standard procedure as described in Fedor et al., Pancreatic Cancer: Methods and Protocols, pp. 89-101. Totowa, N.J.: Human Press, 5 A.D. 0.6 mm sections were prepared from TMA block and re-stained by H&E for each case to confirm the diagnosis.

Histopathologic analysis: All cases from 37 tumor types were graded according to standardized histopathology grading systems. TRIM59 IHC staining signals were assessed by intensity (mostly cytoplasm) and extent (mostly on nucleus) separately. Since in some tumors TRIM59 showed only cytoplasmic staining, for comparing in different tumors, a combined relative score system was used based on both intensity and extent as following: score 1 (intensity/extent) 0/0; score 2: weak/≦25%; score 3: moderate/≦50%; score 4: strong/≧50%. All relative scores were accessed by at least two researchers independently.

### Results

TRIM59 up-regulation in human Renal Cell Carcinoma (RCC) patients: correlation with tumorigenesis and tumor progression until high grade of RCC: Tumour samples from 75 renal cell carcinoma (RCC) patients including all 5 different types of RCC tumors: 43 clear cell carcinoma, 11 papillary renal cell carcinoma, 13 chromophobe renal cell carcinoma, 2 sarcomatoid renal cell carcinoma, and 6 cystic renal cell carcinoma. RCC cases analyzed with Fuhrman grade 1-4 were 4, 38, 28, and 5 respectively. Normal area staining in proximal tubules, or background, endogenous biotin signals were blocked and excluded by additional block reagents (avidin-biotin blocking reagent kit).

TRIM59 IHC staining in tumor areas in RCC was different from cases of CaP-TMA (mainly cytoplasmic). TRIM69-IHC was assessed by visual scoring of both intensity (cytoplasm) and extent (nucleus) microscopically. Correlation of TRIM59 IHC signals by scoring intensity in cytoplasm with grades of all five types of RCC was determined. TRIM59 IHC signals increased with tumor progression from grade 1-3 (p<0.05). All grade 1 tumors (n=4) stained with weak TRIM59 IHC signals, while all highest grade 4 (n=5) tumors showed also weak TRIM59 signals with no or weak nuclear staining. No correlation of TRIM59 IHC staining in nuclear was found, although in low grade RCC showed higher nuclear staining.

TMA analysis of TRIM59 protein expression demonstrates that TRIM59 is a multiple tumor marker: TRIM59 IHC studies was extended to 35-multiple cancer TMA sections (42 tumors, 126 cores, Table 3). Different dilutions (1/300, 1/600 and 1/1200) of TRIM59 antibody were tested.

TABLE 3

| | [symbol] | [symbol] carcinoma | 1 | | |
|---|---|---|---|---|---|
| [symbol] | Number | Metastatic SCC | 1 | [symbol] | 1 |
| 35 tumor-TMA | 42 | [symbol] | | high grade | 2 |
| Prostate TMA | 103 | [symbol] | 1 | BPH | 16 |
| | | | | PIN | 4 |
| Head and neck | 4 | Squamous cell carcinoma | 4 | [symbol] | 4 |
| mucosal tumor | | | | [symbol] | [symbol] |
| | | | | [symbol] | 15 |
| | | | | [symbol] | [symbol] |
| Total | 289 | | | Gleason score 9-10 | 9 |
| | | | | Stroma | 3 |
| | | | | Absent cores | 6 |
| Kidney | 75 | Clear cell carcinoma | 43 | Grade 1 | 4 |
| | | Papillary RCC | 11 | Grade 2 | 38 |
| | | Chromophobe RCC | 13 | Grade 3 | 28 |
| | | Cystic RCC | 6 | Grade 4 | 5 |
| | | Sarcomatoid RCC | 2 | | |
| Bladder | 44 | Urothelial carcinoma | | Low grade | 38 |
| | | | | High grade | 6 |
| Lung | 4 | Bronchoalveolar carcinoma | 1 | Grade 1 | 1 |
| | | Adenocarcinoma | 1 | Grade 2 | 1 |
| | | Large cell carcinoma | 1 | Grade 3 | 1 |
| | | Squamous cell carcinoma | 1 | Grade 4 | 1 |
| Breast | 3 | Invasive lobular carcinoma | 1 | Grade 1 | 1 |
| | | Invasive mammary carcinoma | 2 | Grade 3 | 2 |
| Female Genital | 5 | Endometrial carcinoma | 4 | (Grade 1) | 4 |
| tract | | Ovary, Endometrioid carcinoma | 1 | (Grade2) | 1 |
| Gatrointestinal | 2 | Colon carcinoma | 1 | Low grade | 1 |
| tract | | Pancreas neuroendocrine carcinoma | 1 | high grade | 1 |

[symbol]

indicates data missing or illegible when filed

To further confirm the specificity and reliability of TRIM59 antibody in IHC staining, IHC staining was compared in 35 different tumor-TMA sections with positive (TRIM59 antibody at 1:1200 dilution) and negative controls (no antibody added). As summarized in Table 4, TRIM59 expression was significantly and tissue-specifically up-regulated in most of these 35 tumors. When comparing the relative scores (both intensity and extent) in different tumors, the highest staining was observed in breast, lung, liver, squamous cell carcinoma of skin and endometrial cancers.

TABLE 4
Immunohistochemistry analysis of TRIM59 as a marker in 35 tumor TMA

| | | | | | IHC staining | |
|---|---|---|---|---|---|---|
| | Patient | Core | Pathologic | | Cytoplasm | Nuclear |
| Tumor Type | number | number | Grade | Cell Type | scores | staining |
| Renal clear cell | 2 | 6 | 2 | Epithelial | 2 | — |
| carcinoma, | | | 3 | | 2 | — |
| Adrenal gland cortical | 1 | 3 | N/A | Epithelial | 2-3 | — |
| carcinoma | | | | | | |
| Squamous Cell | 2 | 6 | WD | Epithelial | 3 | — |
| Carcinoma, Skin | | | MD | | 2 | — |
| Basal cell | 2 | 6 | N/A | Epithelial | 2 | — |
| carcinoma, Skin | | | | | 2-3 | |
| Melanoma | 1 | 3 | N/A | Epithelial | 1 | 50% + |
| Endometroid | 2 | 6 | 2 | Epithelial | 2-3 | — |
| adenocarcinoma | | | 1 | | 2-3 | — |
| Leiomyosarcoma | 1 | 3 | N/A | Mesenchymal | 1 | — |
| Omentum serous | 1 | 3 | WD | Epithelial | 1-2 | — |
| adenocarcinoma, | | | | | | |
| Ovary serous | 1 | 3 | N/A | Epithelial | 1-2 | — |
| adenocarcinoma | | | | | | |
| Ovary clear cell | 1 | 3 | PD | Epithelial | 2-3 | 30% + |
| carcinoma | | | | | | |
| Cervix adenocarcinoma | 1 | 3 | WD-MD | Epithelial | 2-3 | — |
| Colon adenocarcinoma | 1 | 3 | Low grade | Epithelial | 1 | — |
| Breast ductal | 1 | 3 | 2/3 | Epithelial | 2-3 | 50% + |
| adenocarcinoma | | | | | | |
| Bladder urothelial | 2 | 6 | Low grade | Epithelial | 1 | — |
| carcinoma | | | 2 (low | Epithelial | 2 | — |
| | | | grade) | | | |
| Stomach GIST | 1 | 3 | | Epithelial | 1-2 | — |
| Esophagus | 1 | 3 | MD | Epithelial | 0-1 | — |
| adenocarcinoma | | | | | | |
| Thyroid, Papillary | 1 | 3 | N/A | Epithelial | 1 | — |
| carcinoma | | | | | | |
| Thyroid, medullary | 1 | 3 | N/A | Epithelial | 2 | — |
| carcinoma | | | | | | |
| Pancreas | 2 | 6 | 2 | Epithelial | 1-2 | — |
| adenocarcinoma, | | | 2 | | 2-3 | — |
| Pancreas endocrine | 1 | 3 | N/A | Epithelial | 3 | — |
| tumor | | | | | | |
| Lung SCC | 1 | 3 | PD | Epithelial | 3 | — |
| Lung mesothelioma | 1 | 3 | MD-PD | | 3 | 20% + |
| Lung adenocarcinoma | 1 | 3 | MD | | 2 | 50% + |
| Lung bronchoalveolar | 1 | 3 | WD | | 2-3 | 50% + |
| carcinoma | | | | | | |
| Lung mesothelioma, | 1 | 3 | MD-PD | | 3 | 20% + |
| biphasic | | | | | | |
| Liver hepatocellular | 1 | 3 | 2/4 | Epithelial | 3 | — |
| carcinoma (HCCa) | | | | | | |
| Liver metastatic | 1 | 3 | N/A | Epithelial | 3 | — |
| carcinoid | | | | | | |
| Small bowel marginal | 1 | 3 | N/A | Lymphocyte | 0-1 | — |
| zone lymphoma | | | | | | |
| Lymph node, follicular | 1 | 3 | 1/3 | Lymphocyte | 0-1 | — |
| lymphoma | | | | | | |
| Lymph node, | 1 | 3 | Low grade | Epithelial | 3 | — |
| metastatic carcinoid | | | | | | |
| Spleen, Hodgkin | 1 | 3 | N/A | Lymphocyte | 1 | — |
| lymphoma | | | | | | |
| Stomach, malt | 1 | 3 | Low grade | Lymphocyte | 0-1 | — |
| lymphoma | | | | | | |
| Thymus invasive | 1 | 3 | N/A | Epithelial | 0-1 | — |
| thymoma | | | | | | |
| Appendix, Goblet cell | 1 | 3 | N/A | Epithelial | 0 | — |
| carcinoid | | | | | | |

Further confirmation of TRIM59 as a tumor marker in patients with eight different tumors: Since the 35 tumor-TMA included only limited cases in each tumor type, additional cases (n=90) of eight different tumor types with all tumor grades were selected, which all showed higher expression of TRIM59 in TMA.

Since some tumors (e.g. prostate) showed mostly cytoplasmic and no nuclear TRIM59-IHC staining, as a comparative study, their relative scores (combine both intensity and extent scores, see Materials and Methods) were assessed. Kidney (RCC, n=75) and prostate cancer (n=25) were used as references and all were assessed by relative scores simultaneously. The highest relative scores were found in SCC of the parotid, mouth, larynx and tongue, followed by lung, breast and female genital tract cancers.

The comparison of relative scores on low and high grades separately was very similar. Cases of grade 1 lung cancer (bronchoalveolar, adenocarcinoma, SCC and large cell carcinoma) and breast cancer (invasive lobular and invasive mammary carcinoma) all showed the strongest staining as compared with other tumors. In endometrial cancer, the TRIM59 relative scores were moderate in grade 1 and moderate to strong in grade 1 and 2. Three types of squamous cell carcinoma (SCC) from mouth, tongue and larynx with different grades also showed relatively high relative scores (both intensity and extent).

1. A method of treating cancer in a mammal comprising the step of inhibiting TRIM59 expression or activity in the mammal.

2. The method of claim 1, comprising administration of a TRIM59 inhibitor to the mammal.

3. The method of claim 2, wherein the inhibitor is an oligonucleotide.

4. The method of claim 3, wherein the oligonucleotide is an antisense oligonucleotide.

5. The method of claim 3, wherein the oligonucleotide is RNA.

6. The method of claim 2, wherein the TRIM59 inhibitor is an antibody.

7. The method of claim 1, wherein the TRIM59 inhibitor is a protein or a chemical inhibitor.

8. The method of claim 1, wherein the cancer is selected from the group consisting of prostate, renal, breast, lung, parotid, gastrointestinal, female genital tract, endometrial, bladder, liver, skin, tongue, mouth, and head and neck mucosal cancer.

9. A method of diagnosing cancer in a mammal comprising determining the expression or activity of TRIM59 in a biological sample, wherein determination of a level of TRIM59 expression or activity that exceeds a baseline value is indicative of cancer in the mammal.

10. The method as defined in claim 9, wherein the determination of a level of TRIM59 expression or activity that exceeds a baseline value is indicative of tumorigenesis.

11. The method as defined in claim 10, comprising the determination of an increased expression level of TRIM59 with phosphorylated serine and threonine residues.

12. The method as defined in claim 9, comprising the determination of an increased expression level of TRIM59 in the nucleus of cells in the sample.

13. The method of claim 9, wherein the cancer is selected from the group consisting of prostate, renal, breast, lung, parotid, gastrointestinal, female genital tract, endometrial, bladder, liver, skin, tongue, mouth, and head and neck mucosal cancer.

14. The method of claim 9, wherein the determination of an increase in the level of phosphorylated tyrosine (pY) TRIM59 in comparison to a baseline level is indicative of an advanced stage of cancer.

15. The method as defined in claim 9, wherein TRIM59 expression is determined immunologically.

16. The method of claim 9, wherein the method includes the additional step of determining in a subsequent biological sample obtained from the mammal the level of TRIM59 expression or activity in comparison with the TRIM59 level of the first biological sample, wherein a determination of a reduced level of TRIM59 expression in the subsequent biological sample as compared with the first sample is indicative of cancer at an advanced stage.

17. A TRIM59 antibody.

18. The antibody of claim 17, to phosphorylated TRIM59.

19. The antibody as defined in claim 18, directed to tyrosine phosphorylated TRIM59.

20. The antibody as defined in claim 18, directed to threonine phosphorylated TRIM59.


SEQUENCE LISTING (59 sequences)

SEQ ID NO: 1 - DNA, 3886 bp, Human
ggcacctgaagcccttcggggcagaggagggcggggactcggggcggctctcagcatccgcctggagctcgtggcgctgtgtttccgtgctgtggagttgcctggtccgcttcctccccgcgaataagaataaaagattctggaggagttggagaagagtgtattcagcccccaaaccacgagatcaacaaagaaatgcacaattttgaggaagagttaacttgtcccatatgttatagtatttttgaagatcctcgtgtactgccatgctctcatacattttgtagaaattgtttggaaaacattcttcaggcatctggtaacttttatatatggagacctttacgaattccactcaagtgccctaattgcagaagtattactgaaattgctccaactggcattgaatctttacctgttaattttgcactaagggctattattgaaaagtaccagcaagaagaccatccagatattgtcacctgccctgaacattacaggcaaccattaaatgtttactgtctattagataaaaaattagtttgtggtcattgccttaccataggtcaacatcatggtcatcctatagatgaccttcaaagtgcctatttgaaagaaaaggacactcctcaaaaactgcttgaacagttgactgacacacactggacagatcttacccatcttattgaaaagctgaaagaacaaaaatctcattctgagaaaatgatccaaggcgataaggaagctgttctccagtattttaaggagcttaatgatacattagaacagaaaaaaaaaagtttcctaacggctctctgtgatgttggcaatctaattaatcaagaatatactccacaaattgaaagaatgaaggaaatacgagagcagcagcttgaattaatggcactgacaatatctttacaagaagagtctccacttaaatttcttgaaaaagttgatgatgtacgccagcatgtacagatcttgaaacaaagaccacttcctgaggttcaacccgttgaaatttatcctcgagtaagcaaaatattgaaagaagaatggagcagaacagaaattggacaaattaagaacgttctcattcccaaaatgaaaatttctccaaaaaggatgtcatgttcctggcctggtaaggatgaaaaggaagttgaatttttaaaaattttaaacattgttgtagttacattaatttcagtaatactgatgtcgatactctttttcaaccaacacatcataacctttttaagtgaaatcactttaatatggttttctgaagcctctctatctgtttaccaaagtttatctaacagtctgcataaggtaaagaatatactgtgtcacattttctatttgttgaaggaatttgtgtggaaaatagtttcccattgaaaatgtcaacctgaattgtttaaatgggcttattctgtacattgctaaacaaaaaatggggtagcatggataaagagcaaactaagctttattagtgctgcaactaatataaacaaatgtttatatttgttgcttcttttggttagcaatgatatgtcaaagttatatctgaaatagtcaaatctttgggaaacagaatctagtaacaatttgaaaagtaatacactatcccatttttattggctttatgatggttgacataatgttgctgtgacatttaaacattcttgtaccaatattgtcttttaccattattatatactgcagttaattggctttacagttctttatatatatagcaaaatcctgaaagaacatatacctttattttgatgtggcttgaagcttttgaatgggtgaataaggatgatagaaaggtttcaaaatcaagcaacaaagtcttaaagtgataaggcatggcttaaagatcttttgatcaaacatacctgtgtttgagatagatttaagagccctaaatgcttatcaccattcactccaaataaaactattgcttttggataactgttagagtaaagtggctttttaaaagaaatttttgagactgggtctcaccttgttgcccaggctggagtgtagttgtctggtcatgactcactgcagtttcgaccacccaggctcaatcgatcctcccgcctctgcctctggagcagctgggacaaggcacacacccccatgcctggctaattaaaaaaattgttttttgtagaaacgaggttttgccatattgcccaggttggtctcaaacttctgggctcaagagatctgcccaccttggcctcccaaagtgctgggattacagacgttagccacactgtgcctgggggccagcattttctaatacttgtcatattctatagtttgtgcaaatttaagattgtttttttttctgctcgtcagtcaaatcagttcttggattaaaaactcattcttattagaacagaatcatgttggtaacttggtctgcaacaggttttgatggcatcatgtggactttattcatcttaactcatttaaattttctaccacattcccttaagctaatgcaaaagtaccaacaacttaatcttttttttttttttttttttttttttttgagacggagtcttgctctgtcgcccaggctggagtgcagtggcgtgatctcggctcattgcaagccccgcctcccgggttcacaccattctcctgcctcagcctcccgagtagctgggactacaggcatctgctaccacgcccagctaattttctgtatttttagtagagatggggtttcactgtgttagccaggatggtctctatctcctgacctcatgatccactcgcctcagcctcccaaaatgctgggattataggcgtgagccgccacgcccggcccaacaacttaatcttttattagctttgcttaagagggccaattaaatcaaagccctttagttccctttaacagggactggagttatgatgcatgtgttacgacttttggcccactgtccatgcaactctaagtgcaggttgatttgctttccagaaatcccaaaggggctgctcttggatcaccgaagagccttacctatatcaaatcaaaaagacattctgggtcagattagactatgctcctggccctacagattgcacataaactatcataaatacagctttttcagggaactagttctaaaactcttacctgctgagaataagtcttaacactaagatgactgtattatatcaatttattattagaatcagacttatacctagcacaattaactattgtgtgggcaaagaacatttaaagggcatagtagaggtaaggagagacacatactcagctagagataaaaatactaagttgcactgattacttaaaatactagtcacaccataaaagtgccctgtagtttcaaaaacacgtaagcaatgaaatgttagccattatgtgttaaactacttaatctcatttctgtgatgtgaatatttttaaccccctttttgtagatgagggaactgacaagtgacttgtccaaggccatatagctgtgagaaaacccatgcattctcttttcagagttcatgctatcactcaactttaaagtaggccaagattaatgttggtaagggtttgtaatctgtaagaatgctaaaaacgtaagtatatatatcattttagatttgacattttgtatcttgccagtttttaggagaacttttcattttgttaagtatgcatgaatatagttgagtatatgagtaactggttcttatgctgctgttttgtatttttaccagcaggaagattgcaaaagttgatgtatgtaaatcttgaaatatttctaagttttatgtataacaaaatatgtattttaataaacttcttttgatattttaaaaaaaaaaa

SEQ ID NO: 2 - protein, 403 aa, Human
MHNFEEELTCPICYSIFEDPRVLPCSHTFCRNCLENILQASGNFYIWRPLRIPLKCPNCRSITEIAPTGIESLPVNFALRAIIEKYQQEDHPDIVTCPEHYRQPLNVYCLLDKKLVCGHCLTIGQHHGHPIDDLQSAYLKEKDTPQKLLEQLTDTHWTDLTHLIEKLKEQKSHSEKMIQGDKEAVLQYFKELNDTLEQKKKSFLTALCDVGNLINQEYTPQIERMKEIREQQLELMALTISLQEESPLKFLEKVDDVRQHVQILKQRPLPEVQPVEIYPRVSKILKEEWSRTEIGQIKNVLIPKMKISPKRMSCSWPGKDEKEVEFLKILNIVVVTLISVILMSILFFNQHIITFLSEITLIWFSEASLSVYQSLSNSLHKVKNILCHIFYLLKEFVWKIVSH

SEQ ID NO: 3 - protein, 402 aa, Mouse
MHNFEEELTCPICYSIFEDPRVLPCSHTFCRNCLENVLQASGNFYIWRPLRIPLKCPNCRSIIEIASTGIESLPVNFALRAIIEKYQQEDHPDVVTCPEHYRQPLNVYCLLDKKLVCGHCLTIGQHHGHPIDDLQSAYLKEKDTPQKLLKQLTDTHWTDITRLIEKLEEQKCHSEKIVQGDKEVVLQYFKELTDTLEQKKKYFLAALCDVGKMINQEYTPQIQGMKEIREQQLELMTITTSLQDESPLKFLEKIDEVRQRVQMLKQRPLPEVQPVEIYPRVSNVLKEEWSRIEIGRIKKAVIPEMRVSSKRTPCSWSDNDEKEMELFKILNIAIVSLISVILMLILLFNHHIITFLNEITSICFSEVFLSVYQSLSKNLYDLNNTVCYTLYLLKEFMWKIVS

SEQ ID NO: 4 - protein, 333 aa, Rat
MHNFEEELTCPICYSIFEDPRVLPCSHTFCRNCLENLQASGNFYIWRPLRIPLKCPNCRSIEIATGIESLPVNFALRAIIEKYQQEDHPDVTCPEHYRQPLNVYCLLDKKLVCGHCLTIGQHHGHPIDDLQSAYLKEKDTPQKLLQLTDTHWTDTLIEKLEQKHSEKQGDKEVLQYFKELDTLEQKKKFLALCDVGINQEYTPQIMKEIREQQLELMTSLQESPLKFLEKDVRQVQLKQRPLPEVQPVEIYPRVSLKEEWSREIGIKIPMSKRCSWDEKEEKILNIVLISVILMILFNHIITFLEITIFSELSVYQSLSLNCYLLKEFWKIVS

SEQ ID NO: 5 - DNA, 19 bp, Artificial
  primer
gaaggtgaaggtcggagtc

SEQ ID NO: 6 - DNA, 20 bp, Artificial
  primer
gaagatggtgatgggatttc

SEQ ID NO: 7 - DNA, 20 bp, Artificial
  primer
tcacctgccctgaacattac

SEQ ID NO: 8 - DNA, 19 bp, Artificial
  primer
cagcttccttatcgccttg

SEQ ID NO: 9 - DNA, 20 bp, Artificial
  primer
ggtctcctctgacttcaaca

SEQ ID NO: 10 - DNA, 54 bp, Artificial
  primer
gatccgaagagtctccacttaaatttcaagagaatttaagtggagactcttctt

SEQ ID NO: 11 - DNA, 55 bp, Artificial
  primer
agcttaagaagagtctccacttaaattctcttgaaatttaagtggagactcttcg

SEQ ID NO: 12 - DNA, 55 bp, Artificial
  primer
gatcctatggttttctgaagcctcttcaagagagaggcttcagaaaaccatatta

SEQ ID NO: 13 - DNA, 54 bp, Artificial
  primer
agcttaatatggttttctgaagcctctctcttgaagaggcttcagaaaaccata

SEQ ID NO: 14 - DNA, 55 bp, Artificial
  primer
gatcctgtcaacctgaattgtttattcaagagataaacaattcaggttgacatta

SEQ ID NO: 15 - DNA, 55 bp, Artificial
  primer
agcttaatgtcaacctgaattgtttatctcttgaataaacaattcaggttgacag

SEQ ID NO: 16 - DNA, 55 bp, Artificial
  primer
gatccatgggcttattctgtacatttcaagagaatgtacagaataagcccattta

SEQ ID NO: 17 - DNA, 55 bp, Artificial
  primer
agcttaaatgggcttattctgtacattctcttgaaatgtacagaataagcccatg

SEQ ID NO: 18 - DNA, 22 bp, Artificial
  primer
gttcacagccattgaaatcccc

SEQ ID NO: 19 - DNA, 22 bp, Artificial
  primer
caaactcagcctcctggcaaag

SEQ ID NO: 20 - DNA, 38 bp, Artificial
  primer
gggttggatccatgcacaattttgaggaggagttaacg

SEQ ID NO: 21 - DNA, 31 bp, Artificial
  primer
gggaaggatccaaggcgagtgatatctgtcc

SEQ ID NO: 22 - DNA, 29 bp, Artificial
  primer
gggttggatcccctcgagtaagcaatgta

SEQ ID NO: 23 - DNA, 30 bp, Artificial
  primer
gggaaggatcctcaacgagaaactattttc

SEQ ID NO: 24 - DNA, 77 bp, Artificial
  primer
gggttaaggagtcctgctttgtcaccatggcacccaagaagaagaggaaggtgcacaattttgaggaggagttaacg

SEQ ID NO: 25 - DNA, 24 bp, Artificial
  primer
cttatcgtcgtcatccttgtaatc

SEQ ID NO: 26 - DNA, 24 bp, Artificial
  primer
cacaattttgaggaggagttaacg

SEQ ID NO: 27 - DNA, 24 bp, Artificial
  primer
atttatcctcgagtaagcaatgta

SEQ ID NO: 28 - DNA, 32 bp, Artificial
  primer
gggttctcgagatctttgtgaaggaaccttac

SEQ ID NO: 29 - DNA, 37 bp, Artificial
  primer
gggaaggtacctctagaatcgatccagacatgataag

SEQ ID NO: 30 - DNA, 18 bp, Artificial
  primer
tggtcttcttgctggtac

SEQ ID NO: 31 - DNA, 18 bp, Artificial
  primer
ggcaacagcgtgtcaaag

SEQ ID NO: 32 - DNA, 23 bp, Artificial
  primer
caagacctagaaggtccattagc

SEQ ID NO: 33 - DNA, 17 bp, Artificial
  primer
ccatcgctttggggagt

SEQ ID NO: 34 - DNA, 16 bp, Artificial
  primer
acaggccggggtttgc

SEQ ID NO: 35 - DNA, 18 bp, Artificial
  primer
ccgaagggaacggaataa

SEQ ID NO: 36 - DNA, 18 bp, Artificial
  primer
ctgggaagccaaggtcat

SEQ ID NO: 37 - DNA, 19 bp, Artificial
  primer
ccttcacgtttgccaactc

SEQ ID NO: 38 - DNA, 17 bp, Artificial
  primer
gcactggtgggctttcc

SEQ ID NO: 39 - DNA, 18 bp, Artificial
  primer
tgaagcccaaggtcaagg

SEQ ID NO: 40 - DNA, 18 bp, Artificial
  primer
caggacggcaaagcagat

SEQ ID NO: 41 - DNA, 19 bp, Artificial
  primer
atgaaggtcctcctgctgc

SEQ ID NO: 42 - DNA, 18 bp, Artificial
  primer
ggggaatcctttgccacc

SEQ ID NO: 43 - DNA, 20 bp, Artificial
  primer
agtgtaagcaacgcacgacg

SEQ ID NO: 44 - DNA, 19 bp, Artificial
  primer
gccaagcaatgacgaaagg

SEQ ID NO: 45 - DNA, 17 bp, Artificial
  primer
gtgcctgaactgtatgc

SEQ ID NO: 46 - DNA, 15 bp, Artificial
  primer
agcccttgggactgg

SEQ ID NO: 47 - DNA, 18 bp, Artificial
  primer
taaagccctaccaaaacc

SEQ ID NO: 48 - DNA, 16 bp, Artificial
  primer
ccccatcatctgcgtc

SEQ ID NO: 49 - DNA, 18 bp, Artificial
  primer
aatggtccaggaaacacg

SEQ ID NO: 50 - DNA, 17 bp, Artificial
  primer
tggctgcaaatcgagaa

SEQ ID NO: 51 - DNA, 18 bp, Artificial
  primer
agacaccgatttcgctac

SEQ ID NO: 52 - DNA, 18 bp, Artificial
  primer
ttttgggacgaggactaa

SEQ ID NO: 53 - DNA, 18 bp, Artificial
  primer
cagcagtagtggaggacg

SEQ ID NO: 54 - DNA, 19 bp, Artificial
  primer
tatgggattcaaaggagtg

SEQ ID NO: 55 - DNA, 19 bp, Artificial
  primer
aagttggggaataggttga

SEQ ID NO: 56 - DNA, 17 bp, Artificial
  primer
aggctttgcagaatgga

SEQ ID NO: 57 - DNA, 21 bp, Artificial
  primer
agaggaccaggagctagagag

SEQ ID NO: 58 - DNA, 21 bp, Artificial
  primer
taatagtcaggagttctcggc

SEQ ID NO: 59 - DNA, 9 bp, Artificial
  FLAG tag
mdykddddk